(19) United States
(12) Patent Application Publication
(10) Pub. No.: US 2017/0239329 A1
(43) Pub. Date: Aug. 24, 2017

(54) THERAPEUTIC COMPOSITION COMPRISING ANNEXIN V

(71) Applicant: ANNEXIN PHARMACEUTICALS AB, Veberöd (SE)

(72) Inventors: Johan FROSTEGÅRD, Stockholm (SE); Anna FROSTEGÅRD, Stockholm (SE); Marianne SVÄRD, Veberöd (SE); Thomas ANDERSSON, Veberöd (SE)

(21) Appl. No.: 15/518,995

(22) PCT Filed: Oct. 15, 2015
(86) PCT No.: PCT/EP2015/073861

§ 371 Date: Apr. 13, 2017

(30) Foreign Application Priority Data
Oct. 15, 2014 (GB) 1418299.2
Oct. 17, 2014 (GB) 1418500.3

(51) Int. Cl. A61K 38/17; A61K 45/06; A61K 9/00; A61K 39/12

(52) U.S. Cl. CPC A61K 38/1709; A61K 39/12; A61K 45/06; A61K 9/0019

(57) ABSTRACT

The present invention provides Annexin A5 for use in a prophylactic or therapeutic method of preventing, reducing, delaying the onset of, or delaying the progression of, direct viral damage to the vascular system and/or immune system, in a subject, wherein the viral infection is caused by a virus selected from the group consisting of (a) a virus capable of causing hemorrhagic fever (VHF), and (b) a virus that presents phosphatidylserine (PS) and mediates cell infection and/or internalisation through PS binding.

### FIELD OF THE INVENTION

The invention relates to novel methods and compositions for protecting the vascular and/or immune system in, and thereby treating, a subject infected or suspected of being infected with, or having been in contact with biological material present in or taken from another subject infected or suspected of being infected with a pathogen, such as a virus or bacteria, capable of causing hemorrhagic fever. Infection with Ebola virus is of particular interest.

### BACKGROUND TO THE INVENTION

The listing or discussion of an apparently prior-published document in this specification should not necessarily be taken as an acknowledgement that the document is part of the state of the art or is common general knowledge.

Hemorrhagic diseases are caused by infection with certain viruses or bacteria. Viruses cause virtually all the hemorrhagic diseases of microbiological origin that arise with any frequency. The various viral diseases are also known as viral hemorrhagic fevers. Bacterial hemorrhagic disease does occur, but rarely. One example of a bacterial hemorrhagic disease is scrub typhus.

Copious bleeding is the hallmark of a hemorrhagic disease. The onset of a hemorrhagic fever or disease can produce mild symptoms that clear up quickly. However, most hemorrhagic diseases are infamous because of the speed that some infections take hold, and the ferocity of their symptoms. Such hemorrhagic maladies, such as Ebola, have high mortality rates.

The viral hemorrhagic (or haemorrhagic) fevers (VHFs) are a diverse group of animal and human illnesses that may be caused by five distinct families of RNA viruses: the families Arenaviridae, Filoviridae, Bunyaviridae, Flaviviridae, and Rhabdoviridae. All types of VHF are characterized by fever and bleeding disorders and all can progress to high fever, shock and death in many cases. Some of the VHF agents cause relatively mild illnesses, such as the Scandinavian nephropathia epidemica (a Hantavirus), while others, such as Ebola virus, can cause severe, life-threatening disease.

Ebola virus infection first appears to disable the immune system (the very system needed to fight the infection) and subsequently disables the vascular system that leads to blood leakage (hemorrhage), hypotension, drop in blood pressure, followed by shock and death. The virus appears to sequentially infect dendritic cells disabling the interferon system (one of the major host anti-viral immune systems) then macrophages (that trigger the formation of blood clots, release of inflammatory proteins and nitric oxide damaging the lining of blood vessels leading to blood leakage) and finally endothelial cells that contribute to blood leakage. The virus also affects organs such as the liver (that dysregulates the formation of coagulation proteins), the adrenal gland (that destroys the ability of the patient to synthesize steroids and leads to circulation failure and disabling of regulators of blood pressure) and the gastro-intestinal tract (leading to diarrhea). The ability of the virus to disable such major mechanisms in the body facilitates the ability of the virus to replicate in an uncontrolled fashion leading to the rapidity by which the virus can cause lethality.

Various laboratories have been working on defining such mechanisms utilizing in vitro culture systems, a variety of animal models including inbred strains of normal and select gene knock out mice, guinea pigs and nonhuman primates that have led to a better understanding of the potential mechanisms involved.

There have also been some advances made in the identification of therapies from the very simple (major supportive type of therapy), to the identification of a number of highly effective chemotherapeutic agents, a variety of highly effective preventive (demonstrating 100% effectiveness in nonhuman primate models) recombinant formulations (adenovirus based, VSV-based, rabies virus based), therapeutic candidate vaccines (cocktail of monoclonal antibodies such as ZMAPP) and alternate approaches (RNAi-based such as TKM-Ebola and antisense based such as AVI-7537).

Examples of chemotherapeutic therapies suggested for the treatment or prevention of Ebola virus include; a) recombinant human activated protein C or therapeutically-functional equivalent thereof; b) recombinant nematode anticoagulant protein c2 (rNAPc2) or therapeutically-functional equivalent thereof; c) a small molecule anti-sense, such as a phosphorodiamidate morpholino oligomers, such as PMOs AVI-6002 and AVI6003, or lipid nanoparticle small interfering RNA, such as LNP-siRNA:TKM-Ebola; d) a broad spectrum nucleoside analog BCX4430 which shows inhibition against a wide variety of viruses including Ebola virus; e) a broad spectrum anti-viral small molecule that inhibits the entry of a wide variety of viruses including Ebola virus by targeting the cathepsin L cleavage of the viral GP, that is required by the virus to fuse with the host cell membrane; f) pyrazinecarboximide derivative T-705 (favipiravir); g) one or more of compounds FGI-103, FGI-104, FGI-106, dUY11, and LJ-001 as described in De Clercq et al, Med. Res. Rev., 2013, 33(6), 1249-1277; h) drugs that target Ebolavirus VP35 and VP40.

The object of the present invention is to identify further suitable therapeutic agents for the treatment of a subject infected or suspected of being infected with a pathogen capable of causing hemorrhagic fever, such as a virus capable of causing hemorrhagic fever (VHF) or a bacteria capable of causing hemorrhagic fever (BHF), or for treating a subject that has been in contact with another subject who is infected or suspected of being infected with a VHF or BHF, or in contact with biological material present in or produced by another subject who is infected or suspected of being infected with a VHF or BHF.

A further particular object of the present invention is to identify suitable therapeutic agents for use in a prophylactic or therapeutic method of preventing, reducing, delaying the onset of, or delaying the progression of, direct viral damage to the vascular system and/or immune system in a subject. For example, it is a further object of the present invention to provide therapies for the direct protection, direct repair and/or direct stabilisation of the vascular system and/or immune system, or the cells thereof, in the subject.

### DESCRIPTION OF THE FIGURES

FIG. 1 shows the cascade of pathological events that results in the rapid severity of Ebolavirus infection.

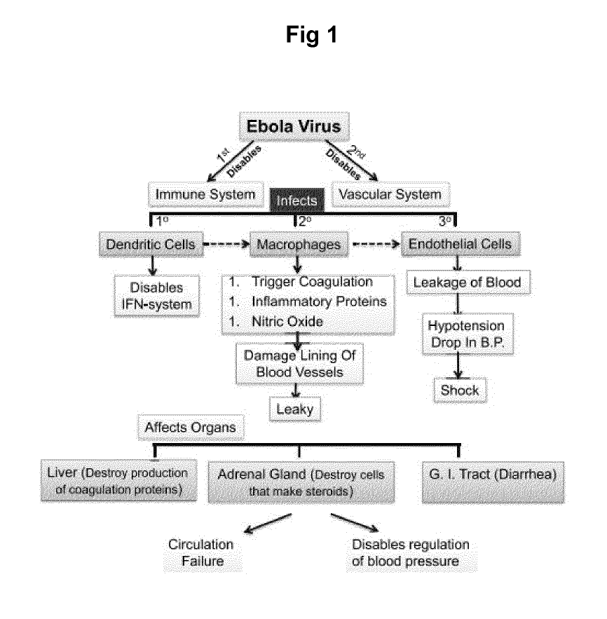

FIG. 2 shows Rift Vallely Fever virus (RVFV) plaque forming units on the monolayer of Vero E6 cells, with and without pre-incubation of RVFV, and culture in the presence of, AnxA5 at various concentrations.

FIG. 3 shows % of inhibition of the formation of RVFV plaque forming units on monolayer of Vero E6 cells following infection with 100 PFU RVFV, compared to a control sample of monolayer of Vero E6 cells that are not incubated with RVFV (the “0 pfu” sample).

### DETAILED DESCRIPTION OF THE INVENTION

A first aspect of the present invention provides Annexin A5 for use in a prophylactic or therapeutic method of preventing, reducing, delaying the onset of, or delaying the progression of, direct viral damage to the vascular system and/or immune system in a subject, wherein the viral infection is caused by a virus selected from the group consisting of—
(a) a virus capable of causing hemorrhagic fever (VHF), and
(b) a virus that presents phosphatidylserine (PS) and mediates cell infection and/or internalisation through PS binding.

That is to say, the first aspect of the present invention provides a prophylactic or therapeutic method of preventing, reducing, delaying the onset of, or delaying the progression of, direct viral damage to the vascular system and/or immune system in a subject, wherein the viral infection is caused by a virus capable of causing hemorrhagic fever (VHF) and/or is a virus that presents phosphatidylserine (PS) and mediates cell infection and/or internalisation through PS binding, the method comprising the administration of a therapeutically effective amount of Annexin A5 to the subject.

To put it yet another way, the first aspect of the present invention provides Annexin A5 for use in the manufacture of a medicament for prophylaxis or therapy by preventing, reducing, delaying the onset of, or delaying the progression of, direct viral damage to the vascular system and/or immune system in a subject, in a subject, wherein the viral infection is caused by a virus capable of causing hemorrhagic fever (VHF) and/or is a virus that presents phosphatidylserine (PS) and mediates cell infection and/or internalisation through PS binding.

In the context of the first aspect of the present invention, Annexin A5 is used for prophylactic or therapeutic method of preventing, reducing, delaying the onset of, or delaying the progression of, direct viral damage to the vascular system and/or immune system in a subject, in contrast to indirect viral damage to the vascular system and/or immune system in a subject. In that context, we include the meaning that Annexin A5 is used to provide a direct action or effect against the cells of the vascular system and/or immune system, rather than providing an indirect action or effect. An example of an indirect action or effect includes preventing, or reducing the rate of, viral infection or transmission in the subject, in particular in cells of the subject which are not cells of the vascular system and/or immune system in a subject. In contrast, the direct action or effect of Annexin A5 in accordance with the first aspect of the present invention can involve direct protection, direct repair and/or direct stabilisation of the vascular system and/or immune system, or the cells thereof, in the subject. The nature of the direct protection, repair or stabilisation may optionally be focussed on the ability of Annexin A5 to prevent, protect against, repair, or stabilise cells in the event of, membrane rupture or cell death in virally-infected cells of the vascular system and/or immune system in the subject.

To put it another way, Annexin A5 may be useful in the context of the aspects of the present invention by providing the inhibition or reduction of endothelial damage and/or activation. Endothelial damage is a common and serious feature in different diseases where infections, both by microorganisms as different types of malaria, and viruses as Ebola and Marburg and other VHFs as descrbed herein represent one common cause, other causes include genetic variants, as in sickle cell anemia, and autoimmune related conditions and yet other, which can be related, include hemolysis. In viral infections, including VHF infectsion such as Ebola, the virus infects dendritic cells, damaging the anti-viral interferon system; macrophages (which triggers formation of blood clots, release of inflammatory factors as interleukin-1 and nitric oxide; and endothelial cells, which could be damaged both by viral factors per se as glycoproteins and by factors as those mentioned above, produced by macrophages. One consequence is thus endothelial dysfunction and damage, and ultimately severe blood leakage (Ansari, J Autoimmun. 2014). In Hemolysis hemoglobin and heme is released into the bloodstream. This condition is a common effect of different conditions, as hemoglobinopathies, autoimmune conditions, bacterial infections, malaria, and trauma and can also be a cause of poisons. Such released hemoglobin and heme have strong effects on endothelial cells including both damage and activation where one cause is oxidative stress. Consequences vasoocclusive events and thrombus formation (Vinchi & Tolosano Oxidative medicine and cellular longevity. 2013; 2013:396527). Another example of endothelial dysfunction and damage (and subsequent vascular disease) where hemolysis could play an important role is in sickle cell anemia. Also here intravascular hemolysis releases heme that activates endothelial TLR4 signaling independent of lipopolyscharide, leading to endothelial damage and vaso-occlusion (Belcher et al, Blood. 2014;123:377-90). In malaria, the hypothesis of the central role of endothelial damage and vascular occlusion is implicated in therapy (Martines et al, J Pathol. 2015, 235(2):153-74; White et al, J Infect Dis. 2013; 208:192-8). Endothelial damage and activation, resulting in symptomatic disease states, may thus be caused by factors as Ebola infection and other infections, both viral and caused by other agents; hemolysis by different mehanisms. The present invention provide Annexin A5 for use in delaying, preventing, protecting against, repairing, and/or stabilising onset of these symptomatic disease states. This can further provide the subject with time to allow for the immune response to gain strength, and also alleviate symptoms.

Accordingly, one embodiment of the first aspect of the present invention can be for preventing, reducing, delaying the onset of, or delaying the progression of, direct viral damage to the vascular system of the subject. For example, Annexin A5 can provide for the direct protection, direct repair and/or direct stabilisation of the vascular system in the subject. Optionally, the Annexin A5 is used to prevent, reduce, delay the onset of, or delay the progression of a one or more vascular system events selected from the group consisting of blood leakage (haemorrhage), hypotension, drop in blood pressure, shock and death in the subject. Further optionally, the Annexin A5 may be useful in the context of the aspects of the prevent invention by preventing, inhibiting or reducing virally-induced vascular inflammation in subjects.

In a further embodiment of the first aspect of the present invention, Annexin A5 can be used for preventing, reducing, delaying the onset of, or delaying the progression of, direct virally-induced nitric oxide damage to the vascular endothelium of the subject.

A second aspect of the present invention provide Annexin A5 for use in a method of prevention, reduction, delaying the onset of, or delaying the progression of, damage, activation, death, and/or disruption to the integrity of, the vascular endothelium or endothelial cells thereof, in a subject infected or suspected of being infected with a virus capable of causing hemorrhagic fever (VHF) as defined above, or in a subject that has been in contact with another subject who is infected or suspected of being infected with the VHF, or in contact with biological material present in or produced by another subject who is infected or suspected of being infected with the VHF. For example, the Annexin A5 may be used in accordance with the second aspect of the present invention for the direct protection, direct repair and/or direct stabilisation of the vascular endothelium or endothelial cells thereof in the subject.

A further embodiment of the first aspect of the present invention provides Annexin A5 for preventing, reducing, delaying the onset of, or delaying the progression of, direct viral damage to the immune system in the subject. For example, in accordance with this embodiment, the viral damage may be selected from damage to the innate immune response, damage to the acquired humoral response, damage to dendritic cells, damage to the regulation of the production of inflammatory factors such as interferon production (including IL1 production), damage to macrophages, and/or damage to monocytes.

In accordance with certain preferred embodiments of the first and/or second aspects of the present invention, the viral infection is caused by a virus capable of causing hemorrhagic fever (VHF), and optionally (a) the method is a method of treating a subject infected or suspected of being infected with a virus capable of causing hemorrhagic fever (VHF); (b) the method is a method of treating a subject that has been in contact with another subject who is infected or suspected of being infected with a VHF; or (c) the method is a method of treating a subject that has been in contact with biological material present in or produced by another subject who is infected or suspected of being infected with a VHF.

The VHF in accordance with the first and/or second aspects of the present invention may, for example, be selected from a virus in family Filoviridae, family Arenaviridae, family Bunyaviridae, family Flaviviridae or family Rhabdoviridae. For example, the VHF may be a virus of family Filoviridae, such as Ebola virus or Marburg virus. Optionally, the VHF is a virus of family Flaviviridae, such as dengue virus.

The virus in accordance with the first and/or second aspects of the present invention may a virus that presents phosphatidylserine (PS) and mediates cell infection and/or internalisation through PS binding, such as an enveloped virus comprising phosphatidylserine (PS) in its envelope. Optionally, the virus mediates cell infection and/or internalisation through binding with a phosphatidylserine-mediated virus entry enhancing receptor (PVEER), such as the T-cell immunoglobulin and mucin 1 (TIM-1) receptor. Further optionally, the virus is selected from the group consisting of a virus in the family Filoviridae (such as Ebola and Marburg); the family Flaviviridae; hepatitis A; alpha viruses; baculoviruses; and arena viruses.

In one preferred embodiment of the first and/or second aspects of the present invention, the VHF is Ebola virus. Accordingly, the present invention also provides Annexin A5 for use in a prophylactic or therapeutic method for preventing, reducing, delaying the onset of, or delaying the progression of, direct Ebola viral damage to the vascular system and/or immune system in a subject in a subject selected from the group consisting of a subject infected or suspected of being infected with Ebola virus, a subject that has been in contact with another subject who is infected or suspected of being infected with Ebola virus, and a subject that has been in contact with biological material present in or produced by another subject who is infected or suspected of being infected with Ebola virus.

In a further embodiment of the first aspect of the present invention, Annexin A5 is provided for use in a prophylactic or therapeutic method, wherein the method (i) prevents, or reduces the rate of, the transmission of a viral infection; (ii) prevents, or protects against, a viral infection; or (iii) treats a viral infection, in a cell type of the subject, selected from the group consisting of epithelial cells, mast cells, B cells, and activated CD4+ cells.

Annexin A5 may optionally be used in accordance with the first and/or second aspects of the present invention wherein the subject is, or is being, treated separately, simultaneously, or sequentially, with one or more chemotherapeutic agents and/or one or more vaccines against the virus. In one preferred embodiment, the Annexin A5 is used as an adjunct therapy in combination (e.g. separately, simultaneously, or sequentially), with one or more chemotherapeutic agents and/or one or more vaccines against the virus. The use of Annexin A5 as an adjunct therapy is discussed in more detail later in this application.

Annexin A5 may optionally be used in accordance with the first and/or second aspects of the present invention wherein the Annexin A5 is formulated in a composition with one or more chemotherapeutic agents and/or one or more vaccines against the virus.

For example, the one or more chemotherapeutic agents against the virus may be optionally selected from

a) recombinant human activated protein C or therapeutically-functional equivalent thereof;

b) recombinant nematode anticoagulant protein c2 (rNAPc2) or therapeutically-functional equivalent thereof;

c) a small molecule anti-sense, such as a phosphorodiamidate morpholino oligomers, such as PMOs AVI-6002 and AVI6003, or lipid nanoparticle small interfering RNA, such as LNP-siRNA:TKM-Ebola;

d) a broad spectrum nucleoside analog BCX4430 which shows inhibition against a wide variety of viruses including Ebola virus;

e) a broad spectrum anti-viral small molecule that inhibits the entry of a wide variety of viruses including Ebolavirus by targeting the cathepsin L cleavage of the viral GP, that is required by the virus to fuse with the host cell membrane;

f) pyrazinecarboximide derivative T-705 (favipiravir);

g) one or more of compounds FGI-103, FGI-104, FGI-106, dUY11, and LJ-001 as described in De Clercq et al, Med. Res. Rev., 2013, 33(6), 1249-1277;

h) drugs that target Ebolavirus VP35 and VP40;

and preferably wherein the virus capable of causing hemorrhagic fever is Ebola virus.

Further optionally, the one or more vaccines against the VHF may be selected from

a) a live-attenuated viral vaccine

b) a killed or inactivated viral vaccine

c) a vaccine comprising viral subunits, and excluding whole live-attenuated, killed or inactivated viruses;

d) a synthetic vaccine;

e) a passive vaccine comprising antibodies capable of providing a vaccine effect against the virus capable of causing hemorrhagic fever, such as antibodies produced in animals (including polyvalent forms and monoclonal antibody forms), and/or sera/immunoglobulins (including polyvalent forms and monoclonal antibody forms) from individuals who have survived from infection with the virus capable of causing hemorrhagic fever, or recombinant antibodies including humanised antibodies and/or therapeutically-active antibody fragments;

and preferably wherein the virus capable of causing hemorrhagic fever is Ebola virus.

In an optional embodiment of the first and/or second aspect of the present invention, the Annexin A5 is administered to the subject within 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the time of infection with the virus (such as the VHF), of the time of contact with biological material present in or taken from another subject infected or suspected of being infected with the virus (such as the VHF), or of time of onset of symptoms characteristic of infection with the virus (such as the VHF).

Optionally, the Annexin A5 can be administered to the subject at a dosage effective to achieve and/or maintain a level of Annexin A5 in the subject's plasma of up to 100 μg/ml, for example, within the range of from 5 to 90 μg/ml, from 10 to 60 μg/ml, from 20 to 50 μg/ml or 30 to 40 μg/ml, from 32 to 38 μg/ml or about from 34 to 36 μg/ml.

Optionally, the Annexin A5 can be administered to the subject with a treatment regime of continuous infusion of Annexin A5 to the subject, or one or more separate administrations, for example, once, twice, three, four or more times daily;

Optionally, the Annexin A5 can be administered to the subject in a dosage amount at each administration in the range of from about 5 to 20 mg/kg patient body weight, such as from about 10 to 15 mg/kg, such as about 11 mg/kg, about 12 mg/kg, about 13 mg/kg or about 14 mg/kg;

Optionally, the Annexin A5 can be administered to the subject at a total doses of Annexin A5 per administration in the region of for example, 0.1 to 3 g, such as 0.2 to 2g, 0.5 to 1.5 g, 0.8 to 1.2 g or about 1g of Annexin A5; and/or

Optionally, the Annexin A5 can be administered to the subject continually, or separate repeated dosages, for a period of at least, or up to, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 day, 4 weeks, 5 weeks, 6 weeks, 7 weeks 8 weeks, 3 months, 4 months, 5 months or longer.

In one preferred option, the Annexin A5 is administered to the subject by injection (such as intravenous injection) or infusion (such as intravenous infusion).

The subject for treatment in accordance with the first and/or second aspects of the present invention may have been in contact with biological material present in or taken from another subject infected or suspected of being infected with, a virus (such as a VHF), and wherein biological material has, or is suspected to have been in contact with an abrasion in the skin of the subject, the mucosal tissue of the subject and/by parenteral exposure to the subject.

The subject for treatment in accordance with the first and/or second aspects of the present invention may have been in contact with biological material present in or taken from another subject infected or suspected of being infected with, a virus (such as a VHF), and the contact occurred within the preceding 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

The subject for treatment in accordance with the first and/or second aspects of the present invention may be a subject suspected of being infected with a virus as defined above, and may or may not display one or more symptoms of infection with the virus.

The subject for treatment in accordance with the first and/or second aspects of the present invention may have been diagnosed with, and have recovered from, an infection with the virus. For example, the subject may have been discharged from hospital, and/or may have stopped displaying one or more symptoms (preferably all symptoms) of infection with the virus. Examples of such symptom are discussed further below. The continued treatment of such patients may be useful in accordance with the present invention since the patient can recover from initial infection but continue to carry inactive virus, which may subsequently reactivate. Continued treatment in accordance with the first or second (or, indeed any other) aspect of the present invention can assist in preventing or reducing the risk or reactivation of the viral infection and/or reduce the impact of reactivation of the viral infection.

Accordingly, in one embodiment of the first or second (or, indeed any other) aspect of the present invention, the subject to be treated may be a subject that has been recovered from an infection with the virus for a period of time of about, up to, or at least, 1, 2, 3, 4, 5, 6 or 7 days, 1, 2, 3 or 4 weeks, 1, 2, 3, 4, 5, 6, 7, 8, 9, 10, 11, or 12 months, 1, 2, 3, 4, 5, 6, 7, 8, 9 or 10 years or more. Optionally, the subject may be treated in accordance with the present invention from the time of the initial infection, or subsequent to the recovery from the initial infection, for example, by chronic administration. Such chromic administration can include regular (e.g. 1, 2, 3, 4, 5, 6, or 7 times per week) administration of Annexin A5 for a period of about, up to, or at least, 1, 2, 3, 4, 5, 6, 7, 8, 9, 10, 11, or 12 months, or 1, 2, 3, 4, 5, 6, 7, 8, 9 or 10 years or more.

The symptoms of infection by VHF (such as Ebola) detectable in the subject may include one or more symptoms selected from initial clinical symptoms, such as excessive or profuse sweating, the onset of fever, myalgia, general malaise, and/or chills; and/or flu-like symptoms optionally accompanied by gastro-intestinal symptoms; maculo-papulary rash, petichae, conjunctival hemorrhage, epistaxis, melena, hematemesis, shock and/or encephalopathy; leukopenia (for example, associated with increased lymphoid cell apoptosis), thrombocytopenia, increased levels of aminotransferase, thrombin and/or partial thromboplastin times, fibrin split products detectable in the blood, and/or disseminated intravascular coagulation (DIC).

Optionally, the subject for treatment in accordance with the first and/or second aspects of the present invention is a human. For example, the human subject may be a health worker, in particular a health worker who works or has worked with patients having, or being suspect of having, an infection with a virus, for example a VHF, such as Ebola virus, or a virus that presents phosphatidylserine (PS) and mediates cell infection and/or internalisation through PS binding. In another option, the human subject may be a family member of, and/or a person that shares or shared accommodation with, a patient having, or being suspect of having, an infection with a virus, for example a VHF, such as Ebola virus, or a virus that presents phosphatidylserine (PS) and mediates cell infection and/or internalisation through PS binding

Alternatively, the subject may be a non-human animal, including an animal selected from the group consisting of dogs, cats, horses, cattle, sheep, pigs, goats, rodents, camels, birds, insects, domesticated animals, and wild animals.

A further aspect of the present invention provides Annexin A5 for use in a prophylactic or therapeutic method of (i) preventing, or reducing the rate of, the transmission of a viral infection; (ii) preventing, or protecting against, a viral infection; or (iii) treating a viral infection, in a subject, wherein the viral infection is caused by a virus selected from the group consisting of—
(a) a virus capable of causing hemorrhagic fever (VHF), and
(b) a virus that presents phosphatidylserine (PS) and mediates cell infection and/or internalisation through PS binding.

Accordingly, in this further aspect, the present invention provides Annexin A5 for use in a prophylactic or therapeutic method of preventing, or reducing the rate of, the transmission of a viral infection, in a subject, wherein the viral infection is caused by a virus capable of causing hemorrhagic fever (VHF).

That is to say, the present invention provides a prophylactic or therapeutic method of preventing, or reducing the rate of, the transmission of a viral infection, in a subject, wherein the viral infection is caused by a virus capable of causing hemorrhagic fever (VHF), the method comprising the administration of a therapeutically effective amount of Annexin A5 to the subject.

To put it yet another way, this aspect provides Annexin A5 for use in the manufacture of a medicament for prophylaxis or therapy by preventing, or reducing the rate of, the transmission of a viral infection, in a subject, wherein the viral infection is caused by a virus capable of causing hemorrhagic fever (VHF).

In another aspect, the present invention provides Annexin A5 for use in a prophylactic or therapeutic method of preventing, or reducing the rate of, the transmission of a viral infection, in a subject, wherein the viral infection is caused by a virus that presents phosphatidylserine (PS) and mediates cell infection and/or internalisation through PS binding.

That is to say, the present invention provides a prophylactic or therapeutic method of preventing, or reducing the rate of, the transmission of a viral infection, in a subject, wherein the viral infection is caused by a virus that presents phosphatidylserine (PS) and mediates cell infection and/or internalisation through PS binding, the method comprising the administration of a therapeutically effective amount of Annexin A5 to the subject.

To put it yet another way, this aspect provides Annexin A5 for use in the manufacture of a medicament for prophylaxis or therapy by preventing, or reducing the rate of, the transmission of a viral infection, in a subject, wherein the viral infection is caused by a virus that presents phosphatidylserine (PS) and mediates cell infection and/or internalisation through PS binding.

In another aspect, the present invention provides Annexin A5 for use in a prophylactic or therapeutic method of preventing, or protecting against, a viral infection, in a subject, wherein the viral infection is caused by a virus capable of causing hemorrhagic fever (VHF).

That is to say, the present invention provides a prophylactic or therapeutic method of preventing, or protecting against, a viral infection, in a subject, wherein the viral infection is caused by a virus capable of causing hemorrhagic fever (VHF), the method comprising the administration of a therapeutically effective amount of Annexin A5 to the subject.

To put it yet another way, this aspect provides Annexin A5 for use in the manufacture of a medicament for prophylaxis or therapy by preventing, or protecting against, a viral infection, in a subject, wherein the viral infection is caused by a virus capable of causing hemorrhagic fever (VHF).

In another aspect, the present invention provides Annexin A5 for use in a prophylactic or therapeutic method of preventing, or protecting against, a viral infection, in a subject, wherein the viral infection is caused by a virus that presents phosphatidylserine (PS) and mediates cell infection and/or internalisation through PS binding.

That is to say, the present invention provides a prophylactic or therapeutic method of preventing, or protecting against, a viral infection, in a subject, wherein the viral infection is caused by a virus that presents phosphatidylserine (PS) and mediates cell infection and/or internalisation through PS binding, the method comprising the administration of a therapeutically effective amount of Annexin A5 to the subject.

To put it yet another way, this aspect provides Annexin A5 for use in the manufacture of a medicament for prophylaxis or therapy by preventing, or protecting against, a viral infection, in a subject, wherein the viral infection is caused by a virus that presents phosphatidylserine (PS) and mediates cell infection and/or internalisation through PS binding.

In another aspect, the present invention provides Annexin A5 for use in a prophylactic or therapeutic method of treating a viral infection, in a subject, wherein the viral infection is caused by a virus capable of causing hemorrhagic fever (VHF).

That is to say, the present invention provides a prophylactic or therapeutic method of treating a viral infection, in a subject, wherein the viral infection is caused by a virus capable of causing hemorrhagic fever (VHF), the method comprising the administration of a therapeutically effective amount of Annexin A5 to the subject.

To put it yet another way, this aspect provides Annexin A5 for use in the manufacture of a medicament for prophylaxis or therapy by treating a viral infection, in a subject, wherein the viral infection is caused by a virus capable of causing hemorrhagic fever (VHF).

In another aspect, the present invention provides Annexin A5 for use in a prophylactic or therapeutic method of treating a viral infection, in a subject, wherein the viral infection is caused by a virus that presents phosphatidylserine (PS) and mediates cell infection and/or internalisation through PS binding.

That is to say, the present invention provides a prophylactic or therapeutic method of treating a viral infection, in a subject, wherein the viral infection is caused by a virus that presents phosphatidylserine (PS) and mediates cell infection and/or internalisation through PS binding, the method comprising the administration of a therapeutically effective amount of Annexin A5 to the subject.

To put it yet another way, this aspect provides Annexin A5 for use in the manufacture of a medicament for prophylaxis or therapy by treating a viral infection, in a subject, wherein the viral infection is caused by a virus that presents phosphatidylserine (PS) and mediates cell infection and/or internalisation through PS binding.

According to a further aspect of the present invention, there is provided Annexin A5 for use in a method of treating a subject infected or suspected of being infected with a pathogen capable of causing hemorrhagic fever, such as a virus capable of causing hemorrhagic fever (VHF) or a bacteria capable of causing hemorrhagic fever (BHF).

To put it another way, this aspect provides a method for treating a subject infected or suspected of being infected with a pathogen capable of causing hemorrhagic fever, such as a virus capable of causing hemorrhagic fever (VHF) or a bacteria capable of causing hemorrhagic fever (BHF), the method comprising the administration of a therapeutically effective amount of Annexin A5 to the subject.

To put it yet another way, this aspect provides Annexin A5 for use in the manufacture of a medicament for treating a subject infected or suspected of being infected with a pathogen capable of causing hemorrhagic fever, such as a virus capable of causing hemorrhagic fever (VHF) or a bacteria capable of causing hemorrhagic fever (BHF).

According to another aspect of the first aspect of the present invention, there is provided Annexin A5 for use in a method of treating a subject that has been in contact with another subject who is infected or suspected of being infected with a pathogen capable of causing hemorrhagic fever, such as a virus capable of causing hemorrhagic fever (VHF) or a bacteria capable of causing hemorrhagic fever (BHF).

To put it another way, this aspect provides a method for treating a subject that has been in contact with another subject who is infected or suspected of being infected with a pathogen capable of causing hemorrhagic fever, such as a virus capable of causing hemorrhagic fever (VHF) or a bacteria capable of causing hemorrhagic fever (BHF), the method comprising the administration of a therapeutically effective amount of Annexin A5 to the subject.

To put it yet another way, this aspect provides Annexin A5 for use in the manufacture of a medicament for treating a subject that has been in contact with another subject who is infected or suspected of being infected with a pathogen capable of causing hemorrhagic fever, such as a virus capable of causing hemorrhagic fever (VHF) or a bacteria capable of causing hemorrhagic fever (BHF).

According to a further aspect of the first aspect of the present invention, there is provided Annexin A5 for use in a method of treating a subject that has been in contact with biological material present in or produced by another subject who is infected or suspected of being infected with a pathogen capable of causing hemorrhagic fever, such as a virus capable of causing hemorrhagic fever (VHF) or a bacteria capable of causing hemorrhagic fever (BHF).

To put it another way, this aspect provides a method for treating a subject that has been in contact with biological material present in or produced by another subject who is infected or suspected of being infected with a pathogen capable of causing hemorrhagic fever, such as a virus capable of causing hemorrhagic fever (VHF) or a bacteria capable of causing hemorrhagic fever (BHF), the method comprising the administration of a therapeutically effective amount of Annexin A5 to the subject.

To put it yet another way, this aspect provides Annexin A5 for use in the manufacture of a medicament for treating a subject that has been in contact with biological material present in or produced by another subject who is infected or suspected of being infected with a pathogen capable of causing hemorrhagic fever, such as a virus capable of causing hemorrhagic fever (VHF) or a bacteria capable of causing hemorrhagic fever (BHF).

In accordance with the foregoing aspects of the present invention, the pathogen capable of causing hemorrhagic fever may be a VHF.

The viral hemorrhagic (or haemorrhagic) fevers (VHFs) are a diverse group of animal and human illnesses that may be caused by at least five distinct families of RNA viruses: the families Arenaviridae, Filoviridae, Bunyaviridae, Flaviviridae, and Rhabdoviridae. All types of VHF may be characterized by fever and bleeding disorders and all can progress to high fever, shock and death in many cases.

A subject who is suspected of being infected with a pathogen capable of causing hemorrhagic fever, such as a virus capable of causing hemorrhagic fever (VHF) or a bacteria capable of causing hemorrhagic fever (BHF) may be a subject with a history of coming into contact with the disease (e.g. by virtue of their employment as a health worker or due to the infection of a family member) and/or may be a subject that displays one or more signs or symptoms of being infected, prior to confirmatory diagnosis.

Signs and symptoms of VHFs characteristically include fever and increased susceptibility to bleeding (bleeding diathesis). Manifestations of VHF often also include flushing of the face and chest, small red or purple spots (petechiae), frank bleeding, swelling caused by edema, low blood pressure (hypotension), and shock. Malaise, muscle pain (myalgia), headache, vomiting, and diarrhea occur frequently. The severity of symptoms varies with the type of virus, with the “VHF syndrome” (capillary leak, bleeding diathesis, and circulatory compromise leading to shock) appearing in a majority of patients with filovirus hemorrhagic fevers (e.g., Ebola and Marburg), CCHF, and the South American hemorrhagic fevers, but in a small minority of patients with dengue, RVF, and Lassa fever.

In accordance with the present invention, the VHF may be Ebola, and subject may display one or more symptoms of Ebola, such as symptoms selected from initial clinical symptoms, such as excessive or profuse sweating, the onset of fever, myalgia, general malaise, and/or chills; and/or flu-like symptoms optionally accompanied by gastro-intestinal symptoms; maculo-papulary rash, petichae, conjunctival hemorrhage, epistaxis, melena, hematemesis, shock and/or encephalopathy; leukopenia (for example, associated with increased lymphoid cell apoptosis), thrombocytopenia, increased levels of aminotransferase, thrombin and/or partial thromboplastin times, fibrin split products detectable in the blood, and/or disseminated intravascular coagulation (DIC).

Definitive diagnosis is usually made at a reference laboratory with advanced biocontainment capabilities. The findings of laboratory investigation vary somewhat between the viruses but in general there is a decrease in the total white cell count (particularly the lymphocytes), a decrease in the platelet count, an increase in the blood serum liver enzymes, and reduced blood clotting ability measured as an increase in both the prothrombin (PT) and activated partial thromboplastin times (PTT). The hematocrit may be elevated. The serum urea and creatine may be raised but this is dependent on the hydration status of the patient. The bleeding time tends to be prolonged.

For example, being a BSL-4 agent, confirmed clinical laboratory diagnosis of viremia during the acute phase of Ebola virus infection is possible with suitable laboratory facilities. The assays that can be utilized are based on the stage of the disease.

During acute disease the assays include a) virus isolation using Vero or Vero E6 cell lines, b) RT-PCR and real time quantitative PCR assays with appropriate false negative and false positive controls, c) antigen capture ELISA, and d) IgM ELISA.

Later during the course of disease the tests that can be utilized include a) IgM and IgG ELISA using authentic viral antigens, and in the case of death, autopsy tissues can be utilized for a) antigen detection using immunostaining techniques, b) immunohistochemical aided detection of Ebola antigen (Zaki et al, J Infect Dis, 1999;179(Suppl. 1):S36e47. , the contents of which are incorporated herein by reference in its entirety), and c) in-situ hybridization techniques for the detection of viral RNA.

The details of each of these techniques have been summarized in Saijo et al, Clin Vaccine Immunol 2006;13:444e51, the contents of which are incorporated herein by reference in its entirety.

The ELISA based assay has been standardized by the CDC for the detection of Ebolavirus specific antibodies. The assay has high sensitivity and has been shown to be capable of detecting antibodies in the sera of humans exposed 10 years previously to Ebola. A cell-based plaque assay and an end point titration assay (TCID50) have also been developed to detect and quantitate filoviruses for use in pre-clinical studies (Shurtleff et al, Viruses 2012;4:3511e30; Smither et al, J Virol Methods 2013;193: 565e71, the contents of which are incorporated herein by reference in their entirety).

For example, the VHF may be a virus in a family selected from Filoviridae, Arenaviridae, Bunyaviridae, Flaviviridae or Rhabdoviridae.

The family Arenaviridae includes the viruses responsible for Lassa fever, Lujo virus, Argentine, Bolivian, Brazilian and Venezuelan hemorrhagic fevers.

The family Bunyaviridae includes the members of the Hantavirus genus that cause hemorrhagic fever with renal syndrome (HFRS), the Crimean-Congo hemorrhagic fever (CCHF) virus from the Nairovirus genus, Garissa virus and Ilesha virus from the Orthobunyavirus and the Rift Valley fever (RVF) virus from the Phlebovirus genus.

The family Filoviridae includes Ebola virus and Marburg virus.

The family Flaviviridae includes dengue, yellow fever, and two viruses in the tick-borne encephalitis group that cause VHF: Omsk hemorrhagic fever virus and Kyasanur Forest disease virus.

The isolation of a member of the Rhabdoviridae responsible for 2 fatal and 2 non-fatal cases of hemorrhagic fever in the Bas-Congo district of the Democratic Republic of Congo has also been reported. The non-fatal cases occurred in healthcare workers involved in the treatment of the other two, suggesting the possibility of person-to-person transmission.

Accordingly, for example in one embodiment of particular interest, the present invention may be applied to viruses in the family Filoviridae, such as Ebola virus and Marburg virus. In another embodiment of particular interest, the present invention may be applied to viruses in the family Flaviviridae, such as dengue virus.

Accordingly, the present invention provides for Annexin A5 for use in a prophylactic or therapeutic method as described above, for (i) preventing, or reducing the rate of, the transmission of an Ebola infection; (ii) preventing, or protecting against, an Ebola infection; or (iii) treating an Ebola infection, in a subject infected or suspected of being infected with Ebola virus, or has been or is expected to be in contact with another subject who is infected or suspected of being infected with Ebola virus, or has been or is expected to be in contact with biological material present in or produced by another subject who is infected or suspected of being infected with Ebola virus.

Accordingly, the present invention provides for Annexin A5 for use in a prophylactic or therapeutic method as described above, for (i) preventing, or reducing the rate of, the transmission of an Marburg infection; (ii) preventing, or protecting against, an Marburg infection; or (iii) treating a Marburg infection, wherein the a subject is infected or suspected of being infected with Marburg virus, or has been or is expected to be in contact with another subject who is infected or suspected of being infected with Marburg virus, or has been or is expected to be in contact with biological material present in or produced by another subject who is infected or suspected of being infected with Marburg virus.

Accordingly, the present invention provides for Annexin A5 for use in a prophylactic or therapeutic method as described above, for (i) preventing, or reducing the rate of, the transmission of an Dengue fever virus infection; (ii) preventing, or protecting against, an Dengue fever virus infection; or (iii) treating a Dengue fever virus infection, wherein the subject is infected or suspected of being infected with Dengue fever virus, or has been or is expected to be in contact with another subject who is infected or suspected of being infected with Dengue fever virus, or has been or is expected to be in contact with biological material present in or produced by another subject who is infected or suspected of being infected with Dengue fever virus.

The present invention also provides Annexin A5 for use in a method a described above, for treating, delaying the onset and/or delaying the progression of infection of the subject by the VHF or BHF.

The present invention also provides Annexin A5 for use in a method a described above for preventing, reducing, delaying the onset of, or delaying the progression of, direct and/or indirect bacterial viral damage, as caused by the BHF or VHF, to the immune and/or vascular system in the subject.

For example, the present invention may be used for preventing, reducing, delaying the onset of, or delaying the progression of, direct and/or indirect bacterial or viral damage to the immune system in the subject, for example, in the context of an Ebola infection. For example, the bacterial or viral damage may be selected from damage to the innate immune response, damage to the acquired humoral response, damage to dendritic cells, damage to the regulation of the production of inflammatory factors such as interferon production (including IL1 production), damage to macrophages, and/or damage to monocytes.

The present invention may be used for preventing, reducing, delaying the onset of, or delaying the progression of, blood leakage (haemorrhage), hypotension, drop in blood pressure, shock or death in the subject.

The present invention may be used for preventing, reducing, delaying the onset of, or delaying the progression of, virally-induced nitric oxide damage to the vascular endothelium of the subject.

The present invention provides Annexin A5 for use in a method of prevention, reduction, delaying the onset of, or delaying the progression of, damage, activation, death, and/or disruption to the integrity of, the vascular endothelium or endothelial cells thereof, in a subject infected or suspected of being infected with a pathogen capable of causing hemorrhagic fever, such as a VHF or BHF. The integrity of the vascular endothelium or endothelial cells thereof may, for example, be determined by the extent of cellular or vascular epithelial leakage and/or by the detection of one or more haemorrhagic events, or the formation of oedema and/or dehydration of the subject.

The present invention provides Annexin A5 for use in a method of prevention, reduction, delaying the onset of, or delaying the progression of, damage, activation, death, and/or disruption to the integrity of, the vascular endothelium or endothelial cells thereof, in a subject that has been or is expected to be in contact with another subject who is infected or suspected of being infected with a pathogen capable of causing hemorrhagic fever, such as a VHF or BHF.

The present invention provides Annexin A5 for use in a method of prevention, reduction, delaying the onset of, or delaying the progression of, damage, activation, death, and/or disruption to the integrity of, the vascular endothelium or endothelial cells thereof, in a subject that has been or is expected to be in contact with biological material present in or produced by another subject who is infected or suspected of being infected with a pathogen capable of causing hemorrhagic fever, such as a VHF or BHF.

A further aspect of the present invention provides for Annexin A5 for use as described above by reference to the various embodiments of the present invention in a prophylactic or therapeutic method, wherein the viral infection is caused by a virus that presents phosphatidylserine (PS) and mediates cell infection and/or internalisation through PS binding. Alternatively, the viral infection may be caused by a virus that presents one or more other types of phospholipids that are bound by Annexin A5 and/or other moieties that are bound by Annexin A5.

Viruses that presents phosphatidylserine (PS) and mediates cell infection and/or internalisation through PS binding can particularly include enveloped viruses comprising phosphatidylserine (PS) in their envelope, especially in the outer layer. The presentation of PS by a virus can be determined by methods known in the art, for example, using an ELISA study to measure the binding of Annexin A5 to the virus. A suitable method can, for example, include the ELISA measurement of haemagglutinin (HA)-tagged Annexin A5 binding to anti-HA antisera, such as described in Moller-Tank, et al, 2013, J. Virol., 87(15), 8327-8341 (the contents of which are incorporated herein by reference).

A group of viruses of particular interest to the present invention includes those which mediate cell infection and/or internalisation through binding with a phosphatidylserine-mediated virus entry enhancing receptor (PVEER). PVEERs are discussed in Moller Tank, et al, 2013, J. Virol., 87(15), 8327-8341, and one example thereof is the T-cell immunoglobulin and mucin 1 (TIM-1) receptor. Further examples may include TIM-4, Gas6 or Protein S/Axl, Mer, and Tyro3, and MFG-E8/integrin αvβ3 or αvβ5

Ebola is an example of one virus of particular interest that presents phosphatidylserine (PS) and mediates cell infection and/or internalisation through PS binding with TIM-1. Moller Tank, et al, 2013, J. Virol., 87(15), 8327-8341.

The present invention recognises that Annexin A5 may be used to inhibit or interrupt the PS-mediated cell infection and/or internalisation of viruses, such as Ebola virus, through PVEERs such as TIM-1, and thereby by can be useful in a prophylactic or therapeutic method of (i) preventing, or reducing the rate of, the transmission of a viral infection; (ii) preventing, or protecting against, a viral infection; or (iii) treating a viral infection, in a subject, wherein the viral infection is caused by a virus that presents phosphatidylserine (PS) and mediates cell infection and/or internalisation through PS binding.

Viruses that presents phosphatidylserine (PS) may, for example, be selected from the group consisting of a virus in the family Filoviridae (such as Ebola and Marburg); the family Flaviviridae; hepatitis A; alpha viruses; baculoviruses; and arena viruses. The viruses may be infectious in, or only in, humans. The viruses may be infectious in, or only in, non-human animals, such as any one or more of animals selected from the group consisting of dogs, cats, cattle, sheep, pigs, goats, rodents, camels, domesticated animals, and wild animals.

PVEERs such as TIM-1, can be involved in the internalisation of viruses into various cell types. In one embodiment, cell types of particular interest for protection and/or treatment in accordance with the present invention may include one or more cell types selected from the group consisting of epithelial cells (including vascular epithelial cells), mast cells, B-cells, and T-cells such as CD4+ cells or CD8+ cells and particularly activated CD4+ cells. TIM-1, also known as HAVCR1 and KIM-1, has been identified as a susceptibility gene for human asthma (McIntire et al, 2003, Nature 425:576). One published amino acid sequence for human TIM-1 protein is shown as:

(SEQ ID NO: 2)
MHPQVVILSLILHLADSVAGSVKVGGEAGPSVTLPCHYSGAVTSMCWRGS
CSLFTCQNGIVWTNGTHVTYRKDTRYKLLGDLSRRDVSLTIENTAVSDSG
VYCCRVEHRGWFNDMKITVSLEIVPPKVTTTPIVTTVPTVTTVRTSTTVP
TTTTVPMTTVPTTTVPTTMSIPTTTTVLTTMTVSTTTSVPTTTSIPTTTS
VPVTTTVSTFVPPMPLPRQNHEPVATSPSSPQPAETHPTTLQGAIRREPT
SSPLYSYTTDGNDTVTESSDGLWNNQTQLFLEHSLLTANTTKGIYAGVCI
SVLVLLALLGVIIAKKYFFKKEVQQLSVSFSSLQIKALQNAVEKEVQAED
NIYIENSLYATD.

TIM-1 is a type I membrane protein with an extracellular region containing an IgV domain, a mucin-rich domain, and a short membrane-proximal stalk containing N-linked glycosylation sites (Ichimura et al, 1998, J. Biol, Chem. 273(7):4135-42). The TIM-1 IgV domain has a disulfide-dependent conformation in which the CC′ loop is folded onto the GFC β strands, resulting in a distinctive cleft formed by the CC′ and FG loops (Santiago et al, 2007, Immunity 26(3):299-310). The cleft built by the CC′ and FG loops is a binding site for phosphatidylserine (Kobayashi et al, 2007, Immunity 27(6):927-40). Antibodies directed to the CC′/FG cleft of the TIM-1 IgV domain inhibit TIM-1 binding to phosphatidylserine and dendritic cells and exhibit therapeutic activity in vivo in a humanized mouse model of allergic asthma (Sonar et al, 2010, J. Clin. Invest. 120: 2767-81).

A further aspect of the present invention is based on the use of Annexin A5 to prevent, inhibit or reduce the ability of the IgV domain of TIM-1, and other PVEERs, from binding to PS presented to it. Annexin A5 also has the ability to bind PS and, in accordance with this aspect of the present invention, is capable of competing with the PVEER to bind to PS.

Accordingly, in one embodiment of this aspect of the present invention, Annexin A5 may be used in a method which inhibits phosphatidylserine binding to TIM-1 (or other PVEER).

For example, this may be prophylactically or therapeutically useful in the context of inhibiting, reducing or preventing the infection of cells with viruses that present phosphatidylserine (PS) and mediate cell infection and/or internalisation through PS.

Alternatively, this may be prophylactically or therapeutically useful in the context of addressing other medical conditions that involve the binding of PS to TIM-1 (or other PVEERs). TIM-1 associated disorders are discussed further below.

Therefore, in another embodiment, the present invention provides a method of inhibiting or reducing binding of TIM-1 or other PVEER, to phosphatidylserine, the method comprising contacting a first cell that expresses TIM- 1 or other PVEER with an amount of Annexin A5 effective to inhibit or reduce binding of the first cell to a second cell that contains phosphatidylserine on its cell surface or to a virus that present phosphatidylserine (PS) on its surface. The method may be an in vivo or in vitro method. In the case of an in vivo method, it may be to treat or prevent a condition that involves the binding of PS to TIM-1 or other PVEER.

In other words, this embodiment of the present invention also provides Annexin A5 for use in a prophylactic or therapeutic method for inhibiting or reducing the binding of TIM-1 or other PVEER, to phosphatidylserine, in a patient in need thereof.

In another embodiment, the present invention provides a method of inhibiting or reducing binding of PS to a TIM-1 or other PVEER on a dendritic cell, the method comprising contacting a dendritic cell that expresses TIM-1 or other PVEER with an amount of Annexin A5 effective to inhibit or reduce binding of PS to the dendritic cell. The method may be an in vivo or in vitro method. In the case of an in vivo method, it may be to treat or prevent a condition that involves the binding of PS to TIM-1 or other PVEER on a dendritic cell.

In other words, this embodiment of the present invention also provides Annexin A5 for use in a prophylactic or therapeutic method for inhibiting or reducing the binding of PS to TIM-1 or other PVEER on a dendritic cell, in a patient in need thereof.

Also disclosed is a method of treating or preventing an inflammatory or autoimmune condition, the method comprising administering to a mammal having an inflammatory or autoimmune condition a pharmaceutical composition comprising a therapeutically effective amount of Annexin A5.

In other words, this embodiment of the present invention also provides Annexin A5 for use in a prophylactic or therapeutic method for preventing, treating or reducing inflammatory or autoimmune condition.

Also disclosed is a method of treating or preventing asthma, the method comprising administering to a mammal having asthma a pharmaceutical composition comprising Annexin A5.

In other words, this embodiment of the present invention also provides Annexin A5 for use in a prophylactic or therapeutic method for preventing, treating or reducing asthma.

Also disclosed is a method of treating or preventing an atopic disorder, the method comprising administering to a mammal having an atopic disorder a pharmaceutical composition comprising a therapeutically effective amount of Annexin A5. The atopic disorder can be, for example, atopic dermatitis, contact dermatitis, urticaria, allergic rhinitis, angioedema, latex allergy, or an allergic lung disorder (e.g., asthma, allergic bronchopulmonary aspergillosis, or hypersensitivity pneumonitis).

In other words, this embodiment of the present invention also provides Annexin A5 for use in a prophylactic or therapeutic method for preventing, treating or reducing an atopic disorder.

Annexin A5 be used as described herein to treat or prevent a variety of TIM-1 associated disorders, and other PVEER-associated disorders, including immunological disorders, such as inflammatory and autoimmune disorders.

The term “treating” includes the meaning of administering a substance or composition described herein in an amount, manner, and/or mode effective to improve a condition, symptom, or parameter associated with a disorder or to prevent progression or exacerbation of the disorder (including secondary damage caused by the disorder) to either a statistically significant degree or to a degree detectable to one skilled in the art.

A subject who is at risk for, diagnosed with, or who has one of these disorders can be administered Annexin A5 in an amount and for a time to provide an overall therapeutic effect. The Annexin A5 can be administered alone (monotherapy) or in combination with other agents (combination therapy), either in admixture or by separate, simultaneous or sequential administration. In the case of a combination therapy, the amounts and times of administration can be those that provide, e.g., an additive or a synergistic therapeutic effect. Further, the administration of the Annexin A5 (with or without the second agent) can be used as a primary, e.g., first line treatment, or as a secondary treatment, e.g., for subjects who have an inadequate response to a previously administered therapy (i.e., a therapy other than one with an Annexin A5).

In one combination therapy embodiment of particular interest, which is applicable to all aspects of the present invention, the Annexin A5 therapy is used to provide direct protection to the vascular and/or immune system, to give the subject protection until another therapeutic regime has time to take effect. That is to say, the Annexin A5 may optionally not be used directly for the purpose of preventing or eradicating an infection, but may be used to maintain (or reduce the deterioration) the subject's health until the other therapeutic regime has time to take effect. To put it another way, Annexin A5 may be used to provide an adjunct therapy, that is, as a secondary treatment that is used together with a primary treatment. As such, the Annexin A5 therapy may be ancillary to a primary treatment, to stabilise a subject in the short-term, and/or to reduce the morbidity and mortality long term. In that case, Annexin A5 may be used as an adjunct therapy by administration to the subject before the onset of the primary therapy and/or during the course of the primary therapy.

Suitable primary therapies include, but are not limited to, the administration of one or more chemotherapeutic agents and/or one or more vaccines against a virus, as described above.

Diseases or conditions treatable with Annexin A5 described herein include, e.g., ischemia-reperfusion injury (e.g., organ ischemia-reperfusion injury such as liver or renal ischemia-reperfusion injury), allergy, asthma, inflammatory bowel disease (IBD), Crohn's disease, transplant rejection, pancreatitis, and delayed type hypersensitivity (DTH).

Additional diseases or conditions treatable with Annexin A5 described herein include, e.g., autoimmune disorders.

Systematic lupus erythromatosis (SLE; lupus) is a TH-2 mediated autoimmune disorder characterized by high levels of autoantibodies directed against intracellular antigens such as double stranded DNA, single stranded DNA, and histones.

Examples of other organ-specific or systemic autoimmune diseases suitable for treatment with Annexin A5 described herein include myasthenia gravis, autoimmune hemolytic anemia, Chagas' disease, Graves disease, idiopathic thrombocytopenia purpura (ITP), Wegener's Granulomatosis, poly-arteritis Nodosa and Rapidly Progressive Crescentic Glomerulonephritis. See, e.g., Benjamini et al.,1996, Immunology, A Short

Course, Third Ed. (Wiley-Liss, New York). In addition, rheumatoid arthritis (RA) is suitable for treatment with Annexin A5 as described herein.

Additional TIM-1 associated diseases or conditions treatable with Annexin A5 described herein include, e.g., Graft-Versus Host Disease (GVHD). GVHD exemplifies a T cell-mediated condition that can be treated using Annexin A5 described herein. GVHD is initiated when donor T cells recognize host antigens as foreign. GVHD, often a fatal consequence of bone marrow transplantation (BMT) in human patients, can be acute or chronic. Acute and chronic forms of GVHD exemplify the development of antigen specific Th1 and Th2 responses, respectively. Acute GVHD occurs within the first two months following BMT, and is characterized by donor cytotoxic T cell-mediated damage to skin, gut, liver, and other organs. Chronic GVHD appears later (over 100 days post-BMT) and is characterized by hyperproduction of immunoglobulin (Ig), including autoantibodies, and damage to the skin, kidney, and other organs caused by Ig-deposition. Nearly 90% of acute GVHD patients go on to develop chronic GVHD. Chronic GVHD appears to be a Th2 T cell mediated disease (De Wit et al, 1993, J. Immunol. 150:361-366). Acute GVHD is a Thl mediated disease (Krenger et al, 1996, Immunol. Res. 15:50-73; Williamson et al, 1996, J. Immunol. 157:689-699). T cell cytotoxicity is a characteristic of acute GVHD. The consequence of donor anti-host cytotoxicity can be seen in various ways. First, host lymphocytes are rapidly destroyed, such that mice experiencing acute GVHD are profoundly immunosuppressed. Second, donor lymphocytes become engrafted and expand in the host spleen, and their cytotoxic activity can be directly measured in vitro by taking advantage of cell lines that express the host antigens that can be recognized (as foreign) by the donor cells. Third, the disease becomes lethal as additional tissues and cell populations are destroyed.

Additional TIM-1 associated diseases or conditions treatable with Annexin A5 described herein include, e.g., atopic disorders. Atopic disorders are characterized by the expression by immune system cells, including activated T cells and APC, of cytokines, chemokines, and other molecules which are characteristic of Th2 responses, such as the

IL-4, IL-5 and IL-13 cytokines, among others. Such atopic disorders therefore will be amenable to treatment with Annexin A5 as described herein. Atopic disorders include airway hypersensitivity and distress syndromes, atopic dermatitis, contact dermatitis, urticaria, allergic rhinitis, angioedema, latex allergy, and an allergic lung disorder (e.g., asthma, allergic bronchopulmonary aspergillosis, and hypersensitivity pneumonitis).

Additional TIM-1 associated diseases or conditions treatable Annexin A5 as described herein include, e.g., numerous immune or inflammatory disorders. Immune or inflammatory disorders include, but are not limited to, allergic rhinitis, autoimmune hemolytic anemia; acanthosis nigricans; Addison's disease; alopecia areata; alopecia universalis; amyloidosis; anaphylactoid purpura; anaphylactoid reaction; aplastic anemia; ankylosing spondylitis; arteritis, cranial; arteritis, giant cell; arteritis, Takayasu's; arteritis, temporal; ataxia-telangiectasia; autoimmune oophoritis; autoimmune orchitis; autoimmune polyendocrine failure; Behcet's disease; Berger's disease; Buerger's disease; bronchitis; bullous pemphigus; candidiasis, chronic mucocutaneous; Caplan's syndrome; post-myocardial infarction syndrome; post-pericardiotomy syndrome; carditis; celiac sprue; Chagas's disease; Chediak-Higashi syndrome; Churg-Strauss disease; Cogan's syndrome; cold agglutinin disease; CREST syndrome; Crohn's disease; cryoglobulinemia; cryptogenic fibrosing alveolitis; dermatitis herpetifomis; dermatomyositis; diabetes mellitus; Diamond-Blackfan syndrome; DiGeorge syndrome; discoid lupus erythematosus; eosinophilic fasciitis; episcleritis; drythema elevatum diutinum; erythema marginatum; erythema multiforme; erythema nodosum; Familial

Mediterranean fever; Felty's syndrome; pulmonary fibrosis; glomerulonephritis, anaphylactoid; glomerulonephritis, autoimmune; glomerulonephritis, post-streptococcal; glomerulonephritis, posttransplantation; glomerulopathy, membranous; Goodpasture's syndrome; granulocytopenia, immune-mediated; granuloma annulare; granulomatosis, allergic; granulomatous myositis; Grave's disease; Hashimoto's thyroiditis; hemolytic disease of the newborn; hemochromatosis, idiopathic; Henoch-Schoenlein purpura; hepatitis, chronic active and chronic progressive; histiocytosis X; hypereosinophilic syndrome; idiopathic thrombocytopenic purpura; Job's syndrome; juvenile dermatomyositis; juvenile rheumatoid arthritis (Juvenile chronic arthritis); Kawasaki's disease; keratitis; keratoconjunctivitis sicca; Landry-Guillain-Barre-Strohl syndrome; leprosy, lepromatous; Loeffler's syndrome; lupus; Lyell's syndrome; lyme disease; lymphomatoid granulomatosis; mastocytosis, systemic; mixed connective tissue disease; mononeuritis multiplex; Muckle-Wells syndrome; mucocutaneous lymph node syndrome; mucocutaneous lymph node syndrome; multicentric reticulohistiocytosis; multiple sclerosis; myasthenia gravis; mycosis fungoides; necrotizing vasculitis, systemic; nephrotic syndrome; overlap syndrome; panniculitis; paroxysmal cold hemoglobinuria; paroxysmal nocturnal hemoglobinuria; pemphigoid; pemphigus; pemphigus erythematosus; pemphigus foliaceus; pemphigus vulgaris; pigeon breeder's disease; polyarteritis nodosa; polymyalgia rheumatic; polymyositis; polyneuritis, idiopathic; Portuguese familial polyneuropathies; pre-eclampsia/eclampsia; primary biliary cirrhosis; progressive systemic sclerosis (scleroderma); psoriasis; psoriatic arthritis; pulmonary alveolar proteinosis; pulmonary fibrosis, Raynaud's phenomenon/syndrome; Reidel's thyroiditis; Reiter's syndrome, relapsing polychrondritis; rheumatic fever; rheumatoid arthritis; sarcoidosis; scleritis; sclerosing cholangitis; serum sickness; Sezary syndrome; Sjogren's syndrome; Stevens-Johnson syndrome; Still's disease; subacute sclerosing panencephalitis; sympathetic ophthalmia; systemic lupus erythematosus; yransplant rejection; ulcerative colitis; undifferentiated connective tissue disease; urticaria, chronic; urticaria, cold; uveitis; vitiligo; Weber-Christian disease; Wegener's granulomatosis, or Wiskott-Aldrich syndrome.

A pharmaceutical composition according to the invention may thus comprise Annexin A5 in admixture with a pharmaceutically or veterinarily acceptable adjuvant, diluent or carrier, which will typically be selected with regard to the intended route of administration and standard pharmaceutical practice. The composition may be in the form of immediate-, delayed- or controlled-release applications. Preferably, the formulation is a unit dosage containing a daily dose or unit, daily sub-dose or an appropriate fraction thereof, of the active ingredient.

The phrases “pharmaceutical or pharmacologically acceptable” refer to compositions that do not produce an adverse, allergic or other untoward reaction when administered to an animal, such as, for example, a human, as appropriate. The preparation of such pharmaceutical compositions are known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, salts, preservatives, drugs, drug stabilizers, excipients, disintegration agents, such like materials and combinations thereof, as would be known to one of ordinary skill in the art. Except insofar as any conventional carrier is incompatible with the active ingredient, its use in the therapeutic or pharmaceutical compositions is contemplated.

The pharmaceutical compositions according to the invention may, or may not, be intended for, and, thus formulated in a manner suitable for, parenteral, intravenous, intra-arterial, intraperitoneal, intra-muscular or subcutaneous administration, or administration from a drug-eluting stent, or they may be administered by infusion techniques. Sterile injectable solutions may be prepared by incorporating the active compounds in the required amount in the appropriate solvent with various of the other ingredients enumerated above, as required, followed by sterilization. The pharmaceutical compositions may be best used in the form of a sterile aqueous solution which may contain other substances, for example, enough salts or glucose to make the solution isotonic with blood. The aqueous solutions may be suitably buffered (preferably to a pH of from 3 to 9), if necessary. The preparation of suitable pharmaceutical formulations under sterile conditions is readily accomplished by standard pharmaceutical techniques well-known to those skilled in the art.

The Annexin A5 may or may not be administered in conjunction with one or more further active agent(s), such as a thrombolytic therapeutic such as aspirin, clopidogrel, ticlopidin, tissue plasminogen activator, urokinase, or a bacterial enzyme such as streptokinase; an analgesic therapeutic such as an opiate, an anti-infective therapeutic such as a beta-lactam, a tetracycline, an amphenicol, or an aminoglycoside. The Annexin A5 may or may not be co-administered with any of one or more further active agent(s), or it may or may not be administered separately, simultaneously or sequentially with such agent(s).

In accordance with the various embodiments of the present invention, the subject may or may not have been, or may or may not be being, treated separately, simultaneously, or sequentially, with one or more chemotherapeutic agents and/or one or more vaccines, such as chemotherapeutic agents and/or vaccines against viruses or other pathogens, including pathogens capable of causing hemorrhagic fever, such as a VHF or BHF, or virus that presents phosphatidylserine (PS) and mediates cell infection and/or internalisation through PS binding.

Further, in accordance with the various embodiments of the present invention, the Annexin A5 used may, or may not, be formulated in a composition with one or more of the above-mentioned chemotherapeutic agents and/or one or more vaccines.

Example of chemotherapeutic agents against viruses in general, or pathogens capable of causing hemorrhagic fever, such as a VHF or BHF, or virus that presents phosphatidylserine (PS) and mediates cell infection and/or internalisation through PS binding, and with particular relevance to Ebola virus, include:
a) recombinant human activated protein C or therapeutically-functional equivalent thereof;
b) recombinant nematode anticoagulant protein c2 (rNAPc2) or therapeutically-functional equivalent thereof;
c) a small molecule anti-sense, such as a phosphorodiamidate morpholino oligomers, such as PMOs AVI-6002 and AVI6003, or lipid nanoparticle small interfering RNA, such as LNP-siRNA:TKM-Ebola;
d) a broad spectrum nucleoside analog BCX4430 which shows inhibition against a wide variety of viruses including Ebola virus;
e) a broad spectrum anti-viral small molecule that inhibits the entry of a wide variety of viruses including Ebolavirus by targeting the cathepsin L cleavage of the viral GP, that is required by the virus to fuse with the host cell membrane ;
f) pyrazinecarboximide derivative T-705 (favipiravir);
g) one or more of compounds FGI-103, FGI-104, FGI-106, dUY11, and LJ-001 as described in De Clercq et al, Med. Res. Rev., 2013, 33(6), 1249-1277;
h) drugs that target Ebolavirus VP35 and VP40;
i) anti-TIM-1 antibodies, such as disclosed in WO 2013/078089 (the contents of which are incorporated herein by reference).

Example of vaccines against viruses and other pathogens in general, such as the pathogen capable of causing hemorrhagic fever, such as a VHF or BHF, or virus that presents phosphatidylserine (PS) and mediates cell infection and/or internalisation through PS binding, and with particular relevance to Ebola virus, include:
a) a live-attenuated viral vaccine
b) a killed or inactivated viral vaccine
c) a vaccine comprising viral subunits, and excluding whole live-attenuated, killed or inactivated viruses;
d) a synthetic vaccine;
e) a passive vaccine comprising antibodies capable of providing a vaccine effect against the virus capable of causing hemorrhagic fever, such as antibodies produced in animals (including polyvalent forms and monoclonal antibody forms), and/or sera/immunoglobulins (including polyvalent forms and monoclonal antibody forms) from individuals who have survived from infection with the virus capable of causing hemorrhagic fever, or recombinant antibodies including humanised antibodies and/or therapeutically-active antibody fragments.

In accordance with the various embodiments of the present invention the Annexin A5 may be administered to the subject within 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the time of infection with the relevant pathogen, such as with the pathogen capable of causing hemorrhagic fever, such as a VHF or BHF, or virus that presents phosphatidylserine (PS) and mediates cell infection and/or internalisation through PS binding.

In accordance with the various embodiments of the present invention the Annexin A5 may be administered to the subject within 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the time of contact with biological material present in or produced by another subject infected or suspected of being infected with a relevant pathogen, such as a pathogen capable of causing hemorrhagic fever, such as a VHF or BHF, or virus that presents phosphatidylserine (PS) and mediates cell infection and/or internalisation through PS binding.

In accordance with the various embodiments of the present invention the Annexin A5 may be administered to the subject within 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the time of onset of symptoms characteristic of infection with a relevant pathogen, such as a pathogen capable of causing hemorrhagic fever, such as a VHF or BHF, or virus that presents phosphatidylserine (PS) and mediates cell infection and/or internalisation through PS binding.

In accordance with the various embodiments of the present invention the Annexin A5 may be administered at a therapeutically effective dosage to treat the subject in a manner as defined above. The skilled person is readily able to determine a suitable dosage in order to achieve the therapeutic effect desired. For example, a suitable dosage may aim to achieve and/or maintain a level of Annexin A5 in the plasma of the subject at greater that naturally-occurring physiological levels of Annexin A5 in the plasma, such as up to 100 μg/ml, for example, within the range of from 5 to 90 μg/ml, from 10 to 60 μg/ml, from 20 to 50 μg/ml or 30 to 40 μg/ml. A plasma level of about from 32 to 38 μg/ml or about from 34 to 36 μg/ml may be suitable.

The treatment regime may, for example, involve the continuous infusion of Annexin A5 to the patient, or can involve one or more administrations, for example, once, twice, three, four or more times daily. For example, administration of Annexin A5 twice daily may be one suitable regime and a dosage amount at each administration in the range of from about 5 to 20 mg/kg patient body weight, such as from about 10 to 15 mg/kg, such as about 11 mg/kg, about 12 mg/kg, about 13 mg/kg or about 14 mg/kg may be one suitable dosage regime. Total doses of Annexin A5 per administration may be in the region of for example, 0.1 to 3 g, such as 0.2 to 2g, 0.5 to 1.5 g, 0.8 to 1.2 g or about 1 g of Annexin A5.

The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

For veterinary use, a compound of the invention is administered as a suitably acceptable formulation in accordance with normal veterinary practice and the veterinary surgeon will determine the dosing regimen and route of administration which will be most appropriate for a particular animal.

The treatment regime may be continued for a therapeutically beneficial period. For example, the Annexin A5 therapy may continued, either by continuous or separate repeated dosages for a period of at least, or up to, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 day, 4 weeks, 5 weeks, 6 weeks, 7 weeks 8 weeks, 3 months, 4 months, 5 months or longer, for example until the subject has made a satisfactory recovery or improvement in condition.

Any suitable route of administration may be used, although parenteral, including injection (such as intravenous, subcutaneous or intramuscular injection), or infusion (such as intravenous infusion) may be particularly suitable.

For example, the Annexin A5 or the functional analogue or variant thereof can be administered parenterally, intravenously, intra-arterially, intra-peritoneally, intra-muscularly, or subcutaneously or locally.

Insofar as the present invention is intended for the treatment of a subject that has been in contact with another subject who is infected or suspected of being infected with a pathogen capable of causing hemorrhagic fever, such as a VHF or BHF or virus that presents phosphatidylserine (PS) and mediates cell infection and/or internalisation through PS binding, or in contact with biological material present in or produced by another subject who is infected or suspected of being infected with a pathogen capable of causing hemorrhagic fever, such as a VHF or BHF or virus that presents phosphatidylserine (PS) and mediates cell infection and/or internalisation through PS binding, then the subject may be one where the contact occurred at the site of an abrasion in the subject's skin, the subject's mucosal tissue and/by parenteral exposure to the subject.

Optionally, the subject to be treated may be one in which contact occurred within the preceding 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the onset of the treatment of the present invention. Such subjects may or may not display symptoms of the infection with hemorrhagic fever, or infection with virus that presents phosphatidylserine (PS) and mediates cell infection and/or internalisation through PS binding, at the time of onset of the therapy of the present invention.

Subjects for treatment in accordance with the present invention are commonly human subjects, although it will be appreciated that the invention can also be applied to the treatment of animal subjects, including for example an animal selected from the group consisting of dogs, cats, cattle, sheep, pigs, goats, rodents, camels, domesticated animals, and wild animals.

A group of humans of particular interest for the treatment of the present invention can include a human health worker, such as an individual who works at a health care facility such as hospital or field hospital, and in particular a health worker who works with patients having, or being suspect of having, an infection with a pathogen capable of causing hemorrhagic fever, such as a VHF or BHF, or virus that presents phosphatidylserine (PS) and mediates cell infection and/or internalisation through PS binding, including Ebola virus.

The Annexin A5 for use in all aspects of the present invention may comprise, consist essentially of, or consist of, a protein having the sequence of human Annexin A5 (SEQ ID NO:1, as shown below), either with or without the N-terminal methionine.

In another embodiment, the Annexin A5 protein may comprise, consist essentially of, or consist of, a variant or mutant of a protein having the sequence of human Annexin A5 (SEQ ID NO:1 1), either with or without the N-terminal methionine. For example, the variant or mutant may differ from SEQ ID NO: 1 (either with, or without, the N-terminal methionine) at any one or more positions, such as at, or up to, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160 or more positions.

The Annexin A5 protein may include one or more of the following:

a) a protein comprising, consisting essentially of, or consisting of the sequence of human Annexin A5 (SEQ ID NO:1);

b) a mammalian orthologue of human Annexin A5;

c) an allelic or genetic variant of a) or b);

d) a functional analogue of Annexin which is a protein which is more than 50%, 60%, 70%, 75%, such as more than 80%, 85%, more than 90%, or even more preferably more than 95% or 99% identical to human Annexin A5, SEQ ID NO:1;

e) a dimer of, or fusion protein comprising, any of a), b), c) or d); and

f) a PEGylated variant of any of a), b), c), d) or e).

In a further embodiment, the Annexin A5 protein may, or may not be, be a fusion protein, which fusion protein comprises, consists essentially of, or consists of: (a) one or more protein sequences comprising the sequence of fusion partner that is/are fused to; (b) one or more protein sequences that comprises, consists essentially of, or consists of, a protein having the sequence of human Annexin A5 (SEQ ID NO:1), either with or without the N-terminal methionine, or a variant, analogue or mutant thereof, or dimer as described above. For example, without limitation, the fusion protein may have a general structure selected from:
in the case of the fusion of two amino acid sequences, for example: H2N-(a)-(b)-COOH; or H2N-(b)-(a)-COOH; or
in the case of the fusion of three amino acid sequences, for example: H2N-(a)-(b)-(a)-COOH; or H2N-(b)-(a)-(b)-COOH; or H2N-(a)-(b)-(b)-COOH; or H2N-(b)-(b)-(a)-COOH; or H2N-(a)-(a)-(b)-COOH; or H2N-(b)-(a)-(a)-COOH; or
in the case of the fusion of four amino acid sequences, for example: H2N-(a)-(a)-(a)-(b)-COOH; or H2N-(a)-(a)-(b)-(a)-COOH; or H2N-(a)-(b)-(a)-(a)-COOH; or H2N-(b)-(a)-(a)-(a)-COOH; or H2N-(a)-(a)-(b)-(b)-COOH; or H2N-(a)-(b)-(a)-(b)-COOH; or H2N-(b)-(a)-(a)-(b)-COO H; or H2N-(a)-(b)-(b)-(a)-COOH; or H2N-(b)-(a)-(b)-(a)-COOH; or H2N-(b)-(b)-(a)-(a)-COOH; or H2N-(b)-(b)-(b)-(a)-COOH; or H2N-(b)-(b)-(a)-(b)-COOH; or H2N-(b)-(a)-(b)-(b)-COOH; or H2N-(a)-(b)-(b)-(b)-COOH; or
in the case of the fusion of five amino acid sequences, for example: or H2N-(a)-(a)-(a)-(a)-(b)-COO H; or H2N-(a)-(a)-(a)-(b)-(a)-COOH; or H2N-(a)-(a)-(b)-(a)-(a)-COOH; or H2N-(a)-(b)-(a)-(a)-(a)-COOH; or H2N-(b)-(a)-(a)-(a)-(a)-COOH; or H2N-(a)-(a)-(a)-(b)-(b)-COOH; or H2N-(a)-(a)-(b)-(a)-(b)-COOH; or H2N-(a)-(b)-(a)-(a)-(b)-COOH; or H2N-(b)-(a)-(a)-(a)-(b)-COO H; or H2N-(a)-(a)-(b)-(b)-(a)-COOH; or H2 N-(a)-(b)-(a)-(b)-(a)-COOH; or H2N-(b)-(a)-(a)-(b)-(a)-COOH; or H2N-(a)-(b)-(b)-(a)-(a)-COOH; or H2N-(b)-(a)-(b)-(a)-(a)-COO H; or H2N-(b)-(b)-(a)-(a)-(a)-COOH; or H2N-(a)-(a)-(b)-(b)-(b)-COOH; or H2N-(a)-(b)-(a)-(b)-(b)-COOH; or H2N-(b)-(a)-(a)-(b)-(b)-COOH; or H2 N-(a)-(b)-(b)-(a)-(b)-COOH; or H2N-(b)-(a)-(b)-(a)-(b)-COOH; or H2N-(b)-(b)-(a)-(a)-(b)-COOH; or H2N-(a)-(b)-(b)-(b)-(a)-COO H; or H2N-(b)-(a)-(b)-(b)-(a)-COOH; or H2N-(b)-(b)-(b)-(a)-(a)-COOH; or H2N-(a)-(b)-(b)-(b)-(b)-COOH; or H2N-(b)-(a)-(b)-(b)-(b)-COOH; or H2N-(b)-(b)-(a)-(b)-(b)-COOH; or H2N-(b)-(b)-(b)-(a)-(b)-COOH; or H2N-(b)-(b)-(b)-(b)-(a)-COOH,
wherein (a) and (b) are as defined above in this paragraph. In the case of multiple fusion partner proteins, as defined by (a), the multiple fusion partners may be same or different. Any fusion partner of interest may be used. For example the fusion partner polypeptide sequence(s) may be suitable to extend the half-life of the molecule within a patient's circulatory system and/or add further functionality to the molecule, such as to add additional therapeutic properties (e.g. anti-coagulant, cell inhibition and/or killing, etc.). In the case of fusion proteins comprising multiple protein sequences having the sequence of human Annexin A5 (SEQ ID NO:1), either with or without the N-terminal methionine, or a variant or mutant thereof, or dimer as described above, as defined by (b), those proteins may be the same or different.

The sequence of a human Annexin A5 protein is defined by SEQ ID NO: 1 as follows—

```
Met Ala Gln Val Leu Arg Gly Thr Val Thr Asp Phe Pro Gly Phe Asp
1 5 10 15
Glu Arg Ala Asp Ala Glu Thr Leu Arg Lys Ala Met Lys Gly Leu Gly
                        20 25 30
Thr Asp Glu Glu Ser Ile Leu Thr Leu Leu Thr Ser Arg Ser Asn Ala
                35 40 45
Gln Arg Gln Glu Ile Ser Ala Ala Phe Lys Thr Leu Phe Gly Arg Asp
        50 55 60
Leu Leu Asp Asp Leu Lys Ser Glu Leu Thr Gly Lys Phe Glu Lys Leu
65 70 75 80
Ile Val Ala Leu Met Lys Pro Ser Arg Leu Tyr Asp Ala Tyr Glu Leu
                                85 90 95
Lys His Ala Leu Lys Gly Ala Gly Thr Asn Glu Lys Val Leu Thr Glu
                        100 105 110
Ile Ile Ala Ser Arg Thr Pro Glu Glu Leu Arg Ala Ile Lys Gln Val
                115 120 125
Tyr Glu Glu Glu Tyr Gly Ser Ser Leu Glu Asp Asp Val Val Gly Asp
        130 135 140
Thr Ser Gly Tyr Tyr Gln Arg Met Leu Val Val Leu Leu Gln Ala Asn
145 150 155 160
Arg Asp Pro Asp Ala Gly Ile Asp Glu Ala Gln Val Glu Gln Asp Ala
                                165 170 175
Gln Ala Leu Phe Gln Ala Gly Glu Leu Lys Trp Gly Thr Asp Glu Glu
                        180 185 190
Lys Phe Ile Thr Ile Phe Gly Thr Arg Ser Val Ser His Leu Arg Lys
              195 200 205
Val Phe Asp Lys Tyr Met Thr Ile Ser Gly Phe Gln Ile Glu Glu Thr
        210 215 220
Ile Asp Arg Glu Thr Ser Gly Asn Leu Glu Gln Leu Leu Leu Ala Val
225 230 235 240
Val Lys Ser Ile Arg Ser Ile Pro Ala Tyr Leu Ala Glu Thr Leu Tyr
                                245 250 255
Tyr Ala Met Lys Gly Ala Gly Thr Asp Asp His Thr Leu Ile Arg Val
                        260 265 270
Met Val Ser Arg Ser Glu Ile Asp Leu Phe Asn Ile Arg Lys Glu Phe
                275 280 285
Arg Lys Asn Phe Ala Thr Ser Leu Tyr Ser Met Ile Lys Gly Asp Thr
        290 295 300
Ser Gly Asp Tyr Lys Lys Ala Leu Leu Leu Leu Cys Gly Glu Asp Asp
305 310 315 320
```

In one aspect of the present invention, the Annexin A5 used in accordance with the present invention consists human Annexin A5, and/or or PEGylated or dimeric forms thereof. That is, in one embodiment, the Annexin A5 is not bound to, conjugated with, or formulated with any other moieties, including active agents. Accordingly, in accordance with one aspect of the present invention, Annexin A5 may be used as the sole active agent in the therapies described above.

In particular embodiments, the functional analogue, mutant or variant of Annexin A5 according to the invention is more than 50%, 60%, 70%, 75%, such as more than 80%, 85%, more than 90%, or even more preferably more than 95% or 99% identical to human Annexin A5, SEQ ID NO:1.

The percent identity between two amino acid sequences is determined as follows. First, an amino acid sequence is compared to, for example, SEQ ID NO:1 using the BLAST 2 Sequences (BI2seq) program from the stand-alone version of BLASTZ containing BLASTN version 2.0.14 and BLASTP version 2.0.14. This stand-alone version of BLASTZ can be obtained from the U.S. government's National Center for Biotechnology

Information web site at ncbi.nlm.nih.gov. Instructions explaining how to use the BI2seq program can be found in the readme file accompanying BLASTZ. BI2seq performs a comparison between two amino acid sequences using the BLASTP algorithm. To compare two amino acid sequences, the options of BI2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences.

The percent identity is determined by dividing the number of matches by the length of the sequence set forth in an identified sequence followed by multiplying the resulting value by 100. For example, if a sequence is compared to the sequence set forth in SEQ ID NO:1 (the length of the sequence set forth in SEQ ID NO:1 is 320) and the number of matches is 288, then the sequence has a percent identity of 90 (i.e., 288÷320*100=90) to the sequence set forth in SEQ ID NO:1.

Thus, a functional analogue, mutant or variant of Annexin A5 may be a protein wherein at one or more positions there have been amino acid insertions, deletions, or substitutions, either conservative or non-conservative, provided that such changes result in a protein whose basic properties to function in an equivalent manner to Annexin A5 have not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr.

Such variants and mutants may be made using the methods of protein engineering and site-directed mutagenesis which are well known in the art.

The functional analogue, mutant or variant of Annexin A5 according to the invention may be a dimer of Annexin A5 (such as DiAnnexin) or a functional analogue or variant thereof, or may be a PEGylated Annexin A5 or a functional analogue or variant thereof. DiAnnexinA5 and PEGylated AnnexinA5 are disclosed in WO 02/067857.

PEGylation is a method well known to those skilled in the art wherein a polypeptide or peptidomimetic compound (for the purposes of the present invention, Annexin A5 or the functional analogue or variant) is modified such that one or more polyethylene glycol (PEG) molecules are covalently attached to the side chain of one or more amino acids or derivatives thereof. It is one of the most important molecule altering structural chemistry techniques (MASC). Other MASC techniques may be used; such techniques may improve the pharmacodynamic properties of the molecule, for example extending its half-life in vivo. A PEG-protein conjugate is formed by first activating the PEG moiety so that it will react with, and couple to, the protein or peptidomimetic compound of the invention. PEG moieties vary considerably in molecular weight and conformation, with the early moieties (monofunctional PEGs; mPEGs) being linear with molecular weights of 12 kDa or less, and later moieties being of increased molecular weights. PEG2, a recent innovation in PEG technology, involves the coupling of a 30 kDa (or less) mPEG to a lysine amino acid (although PEGylation can be extended to the addition of PEG to other amino acids) that is further reacted to form a branched structure that behaves like a linear mPEG of much greater molecular weight (Kozlowski et al., 2001). Methods that may be used to covalently attach the PEG molecules to polypeptides are further described in Roberts et al. (2002) Adv Drug Deliv Rev, 54, 459-476; Bhadra et al. (2002) Pharmazie 57, 5-29; Kozlowski et al. (2001) J Control Release 72, 217-224; and Veronese (2001) Biomaterials 22, 405-417 and references referred to therein.

The advantages of PEGylation include reduced renal clearance which, for some products, results in a more sustained adsorption after administration as well as restricted distribution, possibly leading to a more constant and sustained plasma concentrations and hence an increase in clinical effectiveness (Harris et al. (2001) Clin Pharmacokinet 40, 539-551). Further advantages can include reduced immunogenicity of the therapeutic compound (Reddy (2001) Ann Pharmacother, 34, 915-923), and lower toxicity (Kozlowski et al. (2001), Biodrugs 15, 419-429).

The functional analogue, mutant or variant of Annexin A5 can be a fusion protein comprising the sequence of Annexin A5 or a variant thereof. Thus, for example, Annexin A5 or a variant thereof can be fused to one or more fusion partner polypeptide sequence(s) so as to extend the half-life of the molecule within a patient's circulatory system and/or add further functionality to the molecule. In one embodiment, fusion proteins of Annexin A5 are excluded from the present invention.

A “functional” analogue, mutant or variant of Annexin A5 may be capable of binding to phosphatidylserine on a biological membrane, preferably to a level that is at least 10%, 20%, 30%, 40%, 50%, 60%, 70%, 80%, 90%, 95%, 99% or about 100% of that displayed by human Annexin A5 (SEQ ID NO:1) under the same conditions. A suitable method for measuring Annexin A5 binding to phosphatidylserine on a biological membrane is known in the art (Vermes et al. (1995) J Immuno/Methods, 184(1): p. 39-51).

A “functional” analogue or variant of Annexin A5 may, additionally, or alternatively, possess at least 10%, 20%, 30%, 40%, 50%, 60%, 70%, 80%, 90%, 95%, 99% or about 100% of the therapeutic activity human Annexin A5 (SEQ ID NO:1) when used at the same (i.e. molar equivalent) dosage, for treatment in accordance with the present invention. In this context, the therapeutic activity of a “functional” analogue or variant of Annexin A5 may be determined, compared to that of human Annexin A5 (SEQ ID NO:1), by comparing the ability of a molar equivalent amount of the functional analogue or variant and of human Annexin A5.

A functional analogue or variant of Annexin A5 may, optionally, consist of the sequence of human Annexin A5 (SEQ ID NO:1) with no greater than 50, 40, 30, 20, 10, 5, 4, 3, 2 or 1 consecutive or non-consecutive additional amino acid; and/or no greater than 50, 40, 30, 20, 10, 5, 4, 3, 2 or 1 consecutive or non-consecutive amino acid deletions; and/or no greater than 50, 40, 30, 20, 10, 5, 4, 3, 2 or 1 consecutive or non-consecutive amino acid substitutions.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The term “about” may be used herein to mean a range of ±50%, ±40%, ±30%, ±20%, ±10%, ±5%, ±4%, ±3%, ±2% or ±1% of the value mentioned.

Devices and Kits for Therapy:

Pharmaceutical compositions that include Annexin A5 can be administered with a medical device. The device can designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include Annexin A5, and can be configured to deliver one or more unit doses of the Annexin A5. The device can be further configured to administer a second agent, e.g., a chemo therapeutic agent, either as a single pharmaceutical composition that also includes the Annexin A5 or as two separate pharmaceutical compositions.

The pharmaceutical composition may be administered with a syringe. The pharmaceutical composition can also be administered with a needleless hypodermic injection device, such as the devices disclosed in U.S. Pat. Nos. 5,399, 163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439, 196, which discloses an osmotic drug delivery system having multi-chamber compartments; and U.S. Pat. No. 4,475,196, which discloses an osmotic drug delivery system. Many other devices, implants, delivery systems, and modules are also known.

Annexin A5 can be provided in a kit. In one embodiment, the kit includes (a) a container that contains a composition that includes Annexin A5, and optionally (b) informational material. The informational material can be descriptive, instructional, marketing or other material that relates to the methods described herein and/or the use of the agents for therapeutic benefit.

In an embodiment, the kit also includes a second agent for treating a disorder described herein. For example, the kit includes a first container that contains a composition that includes the Annexin A5, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the Annexin A5, e.g., in a suitable dose, dosage form, or mode of administration (e.g., a dose, dosage form, or mode of administration described herein), to treat a subject in need thereof as described herein. The information can be provided in a variety of formats, include printed text, computer readable material, video recording, or audio recording, or information that provides a link or address to substantive material, e.g., on the internet.

In addition to the Annexin A5, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the Annexin A5 and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

It should be understood that the foregoing description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

Accordingly, the present application provides methods and uses including, but not limited to, the following numbered paragraphs:

1. Annexin A5 for use in a prophylactic or therapeutic method of (i) preventing, or reducing the rate of, the transmission of a viral infection; (ii) preventing, or protecting against, a viral infection; or (iii) treating a viral infection, in a subject, wherein the viral infection is caused by a virus selected from the group consisting of—
(a) a virus capable of causing hemorrhagic fever (VHF), and
(b) a virus that presents phosphatidylserine (PS) and mediates cell infection and/or internalisation through PS binding.

2. Annexin A5 for use in a method according to paragraph 1, wherein the viral infection is caused by a virus capable of causing hemorrhagic fever (VHF).

3. Annexin A5 for use in a method according to paragraph 2, wherein method is a method of treating a subject infected or suspected of being infected with a virus capable of causing hemorrhagic fever (VHF).

4. Annexin A5 for use in a method according to paragraph 2, wherein method is a method of treating a subject that has been in contact with another subject who is infected or suspected of being infected with a VHF.

5. Annexin A5 for use in a method according to paragraph 2, wherein method is a method of treating a subject that has been in contact with biological material present in or produced by another subject who is infected or suspected of being infected with a VHF.

6. Annexin A5 for use in a method according to any of paragraphs 1 to 5, wherein the VHF is selected from a virus in family Filoviridae, family Arenaviridae, family Bunyaviridae, family Flaviviridae or family Rhabdoviridae.

7. Annexin A5 for use in a method according to any of paragraphs 1 to 6, wherein the VHF is a virus of family Filoviridae, such as Ebola virus or Marburg virus.

8. Annexin A5 for use in a method according to any of paragraphs 1 to 6, wherein the VHF is a virus of family Flaviviridae, such as dengue virus.

9. Annexin A5 for use in a method according to any of paragraphs 1 to 8, wherein the method is a prophylactic or therapeutic method for (i) preventing, or reducing the rate of, the transmission of an Ebola infection; (ii) preventing, or protecting against, an Ebola infection; or (iii) treating an Ebola infection, in a subject selected from the group consisting of a subject infected or suspected of being infected with Ebola virus, a subject that has been in contact with another subject who is infected or suspected of being infected with Ebola virus, and a subject that has been in contact with biological material present in or produced by another subject who is infected or suspected of being infected with Ebola virus.

10. Annexin A5 for use in a method according to any of paragraphs 1 to 9, for treating, delaying the onset and/or delaying the progression of infection of the subject by the VHF.

11. Annexin A5 for use in a method according to any of paragraphs 1 to 10, for preventing, reducing, delaying the onset of, or delaying the progression of, direct and/or indirect viral damage to the immune and/or vascular system in the subject.

12. Annexin A5 for use in a method according to paragraph 11, for preventing, reducing, delaying the onset of, or delaying the progression of, direct and/or indirect viral damage to the immune system in the subject, preferably wherein the viral damage is selected from damage to the innate immune response, damage to the acquired humoral response, damage to dendritic cells, damage to the regulation of the production of inflammatory factors such as interferon production (including IL1 production), damage to macrophages, and/or damage to monocytes.

13. Annexin A5 for use in a method according to any of paragraphs 1 to 12, for preventing, reducing, delaying the onset of, or delaying the progression of, blood leakage (haemorrhage), hypotension, drop in blood pressure, shock or death in the subject.

14. Annexin A5 for use in a method according to any of paragraphs 1 to 13, for preventing, reducing, delaying the onset of, or delaying the progression of, directly and/or indirectly virally-induced nitric oxide damage to the vascular endothelium of the subject.

15. Annexin A5 for use in a method of prevention, reduction, delaying the onset of, or delaying the progression of, damage, activation, death, and/or disruption to the integrity of, the vascular endothelium or endothelial cells thereof, in a subject infected or suspected of being infected with a virus capable of causing hemorrhagic fever (VHF) as defined by any of paragraphs 1 to 9, or in a subject that has been in contact with another subject who is infected or suspected of being infected with the VHF, or in contact with biological material present in or produced by another subject who is infected or suspected of being infected with the VHF.

16. Annexin A5 for use in a prophylactic or therapeutic method according to paragraph 1, wherein the viral infection is caused by a virus that presents phosphatidylserine (PS) and mediates cell infection and/or internalisation through PS binding.

17. Annexin A5 for use in a prophylactic or therapeutic method according to paragraph 16, wherein the virus is an enveloped virus comprising phosphatidylserine (PS) in its envelope.

18. Annexin A5 for use in a prophylactic or therapeutic method according to paragraph 16 or 17, wherein the virus mediates cell infection and/or internalisation through binding with a phosphatidylserine-mediated virus entry enhancing receptor (PVEER), such as the T-cell immunoglobulin and mucin 1 (TIM-1) receptor.

19. Annexin A5 for use in a prophylactic or therapeutic method according to any of paragraphs 16 to 18, wherein the virus is selected from the group consisting of a virus in the family Filoviridae (such as Ebola and Marburg); the family Flaviviridae; hepatitis A; alpha viruses; baculoviruses; and arena viruses

20. Annexin A5 for use in a prophylactic or therapeutic method according to any of paragraphs 16 to 19, wherein the method (i) prevents, or reduces the rate of, the transmission of a viral infection; (ii) prevents, or protects against, a viral infection; or (iii) treats a viral infection, in a cell type of the subject, selected from the group consisting of epithelial cells, mast cells, B cells, and activated CD4+cells.

21. Annexin A5 for use in a method according to any of paragraphs 1 to 20, wherein the subject is, or is being, treated separately, simultaneously, or sequentially, with one or more chemotherapeutic agents and/or one or more vaccines against the virus.

22. Annexin A5 for use in a method according to any of paragraphs 1 to 21, wherein the Annexin A5 is formulated in a composition with one or more chemotherapeutic agents and/or one or more vaccines against the virus.

23. Annexin A5 for use in a method according paragraph 21 or 22, wherein the one or more chemotherapeutic agents against the virus are selected from

a) recombinant human activated protein C or therapeutically-functional equivalent thereof;

b) recombinant nematode anticoagulant protein c2 (rNAPc2) or therapeutically-functional equivalent thereof;

c) a small molecule anti-sense, such as a phosphorodiamidate morpholino oligomers, such as PMOs AVI-6002 and AVI6003, or lipid nanoparticle small interfering RNA, such as LNP-siRNA:TKM-Ebola;

d) a broad spectrum nucleoside analog BCX4430 which shows inhibition against a wide variety of viruses including Ebola virus;

e) a broad spectrum anti-viral small molecule that inhibits the entry of a wide variety of viruses including Ebolavirus by targeting the cathepsin L cleavage of the viral GP, that is required by the virus to fuse with the host cell membrane ;

f) pyrazinecarboximide derivative T-705 (favipiravir);

g) one or more of compounds FGI-103, FGI-104, FGI-106, dUY11, and LJ-001 as described in De Clercq et al, Med. Res. Rev., 2013, 33(6), 1249-1277;

h) drugs that target Ebolavirus VP35 and VP40;

and preferably wherein the virus capable of causing hemorrhagic fever is Ebola virus.

24. Annexin A5 for use in a method according paragraph 21 or 22, wherein the one or more vaccines against the VHF are selected from

a) a live-attenuated viral vaccine

b) a killed or inactivated viral vaccine

c) a vaccine comprising viral subunits, and excluding whole live-attenuated, killed or inactivated viruses;

d) a synthetic vaccine;

e) a passive vaccine comprising antibodies capable of providing a vaccine effect against the virus capable of causing hemorrhagic fever, such as antibodies produced in animals (including polyvalent forms and monoclonal antibody forms), and/or sera/immunoglobulins (including polyvalent forms and monoclonal antibody forms) from individuals who have survived from infection with the virus capable of causing hemorrhagic fever, or recombinant antibodies including humanised antibodies and/or therapeutically-active antibody fragments;

and preferably wherein the virus capable of causing hemorrhagic fever is Ebola virus.

25. Annexin A5 for use in a method according to any of paragraphs 1 to 24, wherein the Annexin A5 is administered to the subject within 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the time of infection with the virus (such as the VHF), of the time of contact with biological material present in or taken from another subject infected or suspected of being infected with the virus (such as the VHF), or of time of onset of symptoms characteristic of infection with the virus (such as the VHF).

26. Annexin A5 for use in a method according to any of paragraphs 1 to 25, wherein the Annexin A5 is administered to the subject—
a) at a dosage effective to achieve and/or maintain a level of Annexin A5 in the subject's plasma of up to 100 μg/ml, for example, within the range of from 5 to 90 μg/ml, from 10 to 60 μg/ml, from 20 to 50 μg/ml or 30 to 40 μg/ml, from 32 to 38 μg/ml or about from 34 to 36 μg/ml;
b) with a treatment regime of continuous infusion of Annexin A5 to the subject, or one or more separate administrations, for example, once, twice, three, four or more times daily;
c) is administered in a dosage amount at each administration in the range of from about 5 to 20 mg/kg patient body weight, such as from about 10 to 15 mg/kg, such as about 11 mg/kg, about 12 mg/kg, about 13 mg/kg or about 14 mg/kg;
d) at a total doses of Annexin A5 per administration in the region of for example, 0.1 to 3 g, such as 0.2 to 2g, 0.5 to 1.5 g, 0.8 to 1.2 g or about 1g of Annexin A5; and/or
e) continually, or separate repeated dosages, for a period of at least, or up to, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 day, 4 weeks, 5 weeks, 6 weeks, 7 weeks 8 weeks, 3 months, 4 months, 5 months or longer.

27. Annexin A5 for use in a method according to any of paragraphs 1 to 26, wherein the Annexin A5 is administered to the subject by injection (such as intravenous injection) or infusion (such as intravenous infusion).

28. Annexin A5 for use in a method according to any of paragraphs 1 to 27, wherein the subject has been in contact with biological material present in or taken from another subject infected or suspected of being infected with, a virus (such as a VHF), and wherein biological material has, or is suspected to have been in contact with an abrasion in the skin of the subject, the mucosal tissue of the subject and/by parenteral exposure to the subject.

29. Annexin A5 for use in a method according to any of paragraphs 1 to 28, wherein the subject has been in contact with biological material present in or taken from another subject infected or suspected of being infected with, a virus (such as a VHF), and the contact occurred within the preceding 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

30. Annexin A5 for use in a method according to any of paragraphs 1 to 29 wherein the subject to be treated is a subject suspected of being infected with a virus as defined by any one of paragraphs 1 to 9 or paragraphs 16 to 19.

31. Annexin A5 for use in a method according to paragraph 30 wherein the subject suspected of being infected displays one or more symptoms of infection with the virus.

32. Annexin A5 for use in a method according to any of paragraphs 1 to 31, wherein the symptoms of infection by VHF (such as Ebola) detectable in the subject include one or more symptoms selected from initial clinical symptoms, such as excessive or profuse sweating, the onset of fever, myalgia, general malaise, and/or chills; and/or flu-like symptoms optionally accompanied by gastro-intestinal symptoms; maculo-papulary rash, petichae, conjunctival hemorrhage, epistaxis, melena, hematemesis, shock and/or encephalopathy; leukopenia (for example, associated with increased lymphoid cell apoptosis), thrombocytopenia, increased levels of aminotransferase, thrombin and/or partial thromboplastin times, fibrin split products detectable in the blood, and/or disseminated intravascular coagulation (DIC).

33. Annexin A5 for use in a method according to any of paragraphs 1 to 32, wherein the subject is a human, or a non-human animal, including an animal selected from the group consisting of dogs, cats, horses, cattle, sheep, pigs, goats, rodents, camels, birds, insects, domesticated animals, and wild animals.

34. Annexin A5 for use in a method according to any of paragraphs 1 to 33, wherein the subject is a human, such as a human health worker, in particular a health worker who works or has worked with patients having, or being suspect of having, an infection with a virus, for example a VHF, such as Ebola virus, or a virus that presents phosphatidylserine (PS) and mediates cell infection and/or internalisation through PS binding.

35. Annexin A5 for use in a method according to any of paragraphs 1 to 34, wherein the subject is a family member of, and/or shares or shared accommodation with, a patient having, or being suspect of having, an infection with a virus, for example a VHF, such as Ebola virus, or a virus that presents phosphatidylserine (PS) and mediates cell infection and/or internalisation through PS binding.

The present invention will now be described with reference to a non-limiting example.

The following example is included to demonstrate particular embodiments of the invention. It should be appreciated by those of skill in the art that the techniques disclosed in the example which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Methodology

The anti-viral effect of Annexin A5 (ANXAS) has been studied by using a Plaque Reduction Neutralization Test (PRNT) for an exemplary viral hemorrhagic fever, the Bunyaviridae family member Rift Valley fever virus (RVFV).

The study investigates the effect of ANXAS at different concentrations (0.1, 1, 10, 25 and 50 μg/mL) against a concentration of 100 plaque forming units (PFU) of RVFV. Blank buffer, or buffer containing ANXAS at the selected concentration, was first preincubated with RVFV during the attachment phase (viral binding to cells). The selected concentration of ANXAS was also present during the infectious phase by adding it at the selected concentration into a carboxy methyl cellulose (CMC) overlay.

African Green monkey Vero E6 cells grown in DMEM+Glutamax (high glucose) were used for the PRNT and is the standardised cell line commonly used to study viral (including) Ebola infections.

Different concentrations of ANXA5 in buffer were preincubated for 90 min. with a virus suspension containing approximately 100 PFU of RVFV or, in the case of controls, no pre-incubation in the “100 pfu” sample or pre-incubation with blank buffer in the “buffer” sample, before adsorption to a confluent monolayer of Vero E6 cells in e.g. 6-well or 12-well plates. After an adsorption period of 60 min and subsequent washing, the cells were covered with an overlay containing 1% CMC prepared in CMC media (containing the corresponding concentration of ANXA5) and incubated for six days at 37 ° C./5% CO₂.

After fixation with 10% formaldehyde prepared in PBS, the plaques were visualized by counterstaining with 1% crystal violet in water containing 20% ethanol and 0.7% NaCl and counted after destaining with tap water.

The plaque forming units were calculated manually on a light table.

### References

Näaslund J., Lagerqvist N., Lundkvist Å., Evander M., Ahlm C. and Bucht G. (2008). Kinetics of Rift Valley Fever Virus in experimentally infected mice using quantitative real-time RT-PCR. J. Virol. Methods. 151:277-82

Lagerqvist N., Näslund J., Lundkvist Å., Bouloy M., Ahlm C. and Bucht G. (2009). Characterisation of immune responses and protective efficacy in mice after immunisation with Rift Valley Fever virus cDNA constructs. Virology Journal. Jan 17;6:6.

Näslund J., Lagerqvist N., Ahlm C., Weber F. and Bucht G. (2009). Vaccination with Rift Valley Fever virus-like particles protects mice from lethal wild type virus infection. Virology. 15;385(2):409-415

Näslund J., Kernerb A, Drobnia, P, Bucht G., Evander M., Ahlm C. (2011). Detection of Puumala and Rift Valley Fever virus by quantitative RT-PCR and virus viability tests in samples of blood dried and stored on filter paper. J. Virol. Methods. 178:186-190

Cecilia Engdahl, Jonas Näslund, Lena Lindgren, Clas Ahlm, Göran Bucht. (2012). The Rift Valley Fever Virus Protein NSm and Putative Cellular Protein Interactions. Virol J. 2012. 28;9:139

### Results

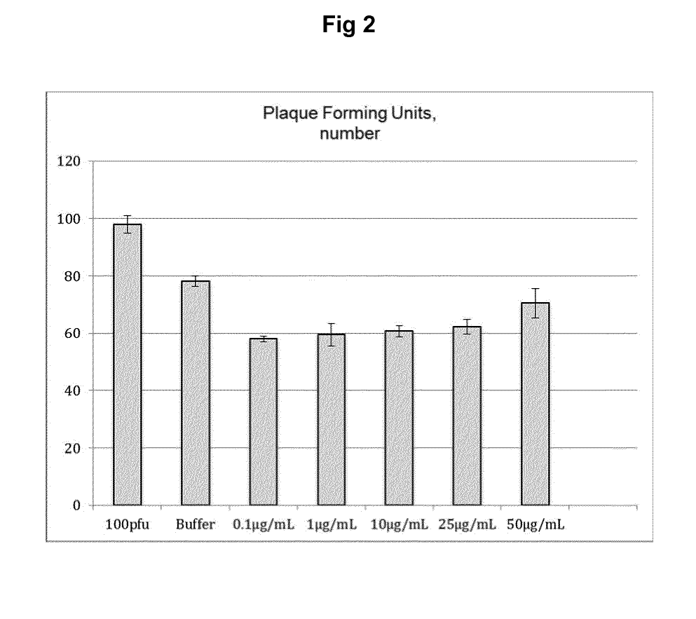

FIG. 2 shows number of RVFV plaque forming units on the monolayer of Vero E6 cells. Pre-incubation of RVFV with AnxA5 at various concentrations reduces the number of plaque forming units formed compared to the control.

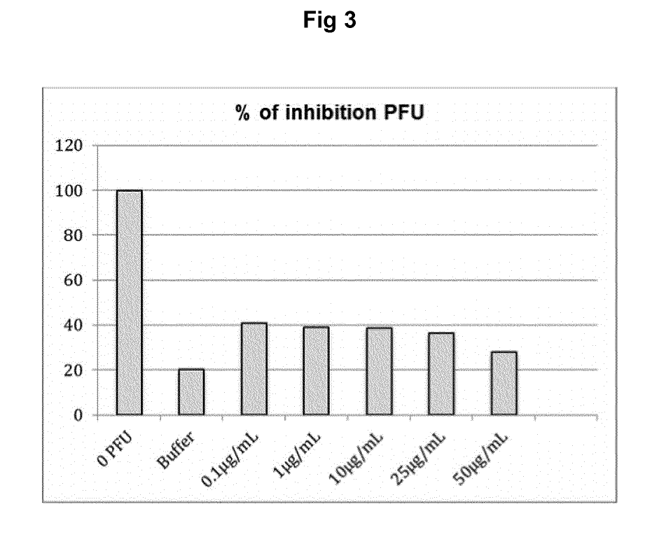

FIG. 3 shows % of inhibition of the formation of RVFV plaque forming units on monolayer of Vero E6 cells following infection with 100 pfu RVFV, compared to a control sample of monolayer of Vero E6 cells that are not incubated with RVFV (the “0 pfu” sample).

### Conclusions

The results of this study show that cell entry of an exemplary viral hemorrhagic fever, RVFV, is reduced by pre-incubation with AnxA5, as indicated by a reduction in plaque numbers.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of preventing, reducing, delaying the onset of, or delaying the progression of, direct viral damage to the vascular system and/or immune system in a subject comprising administering to said subject Annexin A5, wherein the viral infection is caused by a virus selected from the group consisting of—
(a) a virus capable of causing hemorrhagic fever (VHF), and
(b) a virus that presents phosphatidylserine (PS) and mediates cell infection and/or internalization through PS binding.

2. The method according to claim 1 preventing, reducing, delaying the onset of, or delaying the progression of, direct viral damage to the vascular system of the subject.

3. The method according to claim 2, for the direct protection, direct repair and/or direct stabilisation of the vascular system in the subject.

4. The method according to claim 1, for preventing, reducing, delaying the onset of, or delaying the progression of, blood leakage (haemorrhage), hypotension, drop in blood pressure, shock or death in the subject.

5. The method according to claim 1, for preventing, reducing, delaying the onset of, or delaying the progression of, direct virally-induced nitric oxide damage to the vascular endothelium of the subject.

6. A method of prevention, reduction, delaying the onset of, or delaying the progression of, damage, activation, death, and/or disruption to the integrity of, the vascular endothelium or endothelial cells thereof, in a subject infected or suspected of being infected with a virus capable of causing hemorrhagic fever (VHF), or in a subject that has been in contact with another subject who is infected or suspected of being infected with the VHF, or in contact with biological material present in or produced by another subject who is infected or suspected of being infected with the VHF, comprising administering to said subject Annexin A5.

7. The method according to claim 6, for the direct protection, direct repair and/or direct stabilization of the vascular endothelium or endothelial cells thereof in the subject.

8. The method according to claim 1, preventing, reducing, delaying the onset of, or delaying the progression of, direct viral damage to the immune system in the subject.

9. The method according to claim 8, wherein the viral damage is selected from damage to the innate immune response, damage to the acquired humoral response, damage to dendritic cells, damage to the regulation of the production of inflammatory factors such as interferon production (including IL1 production), damage to macrophages, and/or damage to monocytes.

10. The method according to claim 1, wherein the viral infection is caused by a virus capable of causing hemorrhagic fever (VHF).

11. The method according to claim 10, wherein method is a method of treating a subject infected or suspected of being infected with a virus capable of causing hemorrhagic fever (VHF).

12. The method according to claim 10, wherein method is a method of treating a subject that has been in contact with another subject who is infected or suspected of being infected with a VHF.

13. The method according to claim 10, wherein method is a method of treating a subject that has been in contact with biological material present in or produced by another subject who is infected or suspected of being infected with a VHF.

14. The method according to claim 1, wherein the VHF is selected from a virus in family Filoviridae, family Arenaviridae, family Bunyaviridae, family Flaviviridae or family Rhabdoviridae.

15. The method according to claim 14, wherein the VHF is Ebola virus or Marburg virus.

16. The method according to claim 14, wherein the VHF is a virus of family Flaviviridae, such as dengue virus.

17. The method according to claim 1, wherein the method is a prophylactic or therapeutic method for preventing, reducing, delaying the onset of, or delaying the progression of, direct Ebola viral damage to the vascular system and/or immune system in a subject in a subject selected from the group consisting of a subject infected or suspected of being infected with Ebola virus, a subject that has been in contact with another subject who is infected or suspected of being infected with Ebola virus, and a subject that has been in contact with biological material present in or produced by another subject who is infected or suspected of being infected with Ebola virus.

18. The method according to claim 1, wherein the viral infection is caused by a virus that presents phosphatidylserine (PS) and mediates cell infection and/or internalization through PS binding.

19. The method according to claim 18, wherein the virus is an enveloped virus comprising phosphatidylserine (PS) in its envelope.

20. The method according to claim 18, wherein the virus mediates cell infection and/or internalisation through binding with a phosphatidylserine-mediated virus entry enhancing receptor (PVEER), such as the T-cell immunoglobulin and mucin 1 (TIM-1) receptor.

21. The method according to any of claim 18, wherein the virus is selected from the group consisting of a virus in the family Filoviridae (such as Ebola and Marburg); the family Flaviviridae; hepatitis A; alpha viruses; baculoviruses; and arena viruses

22. The method according to claim 18, wherein the method (i) prevents, or reduces the rate of, the transmission of a viral infection; (ii) prevents, or protects against, a viral infection; or (iii) treats a viral infection, in a cell type of the subject, selected from the group consisting of epithelial cells, mast cells, B cells, and activated CD4+ cells.

23. The method according to claim 1, wherein the subject is, or is being, treated separately, simultaneously, or sequentially, with one or more chemotherapeutic agents and/or one or more vaccines against the virus.

24. The method according to claim 1, wherein the Annexin A5 is formulated in a composition with one or more chemotherapeutic agents and/or one or more vaccines against the virus.

25. The method according claim 23, wherein the one or more chemotherapeutic agents against the virus are selected from:
a) recombinant human activated protein C or therapeutically-functional equivalent thereof;
b) recombinant nematode anticoagulant protein c2 (rNAPc2) or therapeutically-functional equivalent thereof;
c) a small molecule anti-sense, such as a phosphorodiamidate morpholino oligomers, such as PMOs AVI-6002 and AVI6003, or lipid nanoparticle small interfering RNA, such as LNP-siRNA:TKM-Ebola;
d) a broad spectrum nucleoside analog BCX4430 which shows inhibition against a wide variety of viruses including Ebola virus;
e) a broad spectrum anti-viral small molecule that inhibits the entry of a wide variety of viruses including Ebolavirus by targeting the cathepsin L cleavage of the viral GP, that is required by the virus to fuse with the host cell membrane ;
f) pyrazinecarboximide derivative T-705 (favipiravir);
g) one or more of compounds FGI-103, FGI-104, FGI-106, dUY11, and LJ-001 as described in De Clercq et al, Med. Res. Rev., 2013, 33(6), 1249-1277;
h) drugs that target Ebolavirus VP35 and VP40;
and preferably wherein the virus capable of causing hemorrhagic fever is Ebola virus.

26. The method according claim 23, wherein the one or more vaccines against the VHF are selected from:
a) a live-attenuated viral vaccine
b) a killed or inactivated viral vaccine
c) a vaccine comprising viral subunits, and excluding whole live-attenuated, killed or inactivated viruses;
d) a synthetic vaccine;
e) a passive vaccine comprising antibodies capable of providing a vaccine effect against the virus capable of causing hemorrhagic fever, such as antibodies produced in animals (including polyvalent forms and monoclonal antibody forms), and/or sera/immunoglobulins (including polyvalent forms and monoclonal antibody forms) from individuals who have survived from infection with the virus capable of causing hemorrhagic fever, or recombinant antibodies including humanised antibodies and/or therapeutically-active antibody fragments;
and preferably wherein the virus capable of causing hemorrhagic fever is Ebola virus.

27. The method according to claim 1, wherein the Annexin A5 is administered to the subject within 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the time of infection with the virus (such as the VHF), of the time of contact with biological material present in or taken from another subject infected or suspected of being infected with the virus (such as the VHF), or of time of onset of symptoms characteristic of infection with the virus (such as the VHF).

28. The method according to claim 1, wherein the Annexin A5 is administered to the subject—
a) at a dosage effective to achieve and/or maintain a level of Annexin A5 in the subject's plasma of up to 100 μg/ml, for example, within the range of from 5 to 90 μg/ml, from 10 to 60 μg/ml, from 20 to 50 μg/ml or 30 to 40 μg/ml, from 32 to 38 μg/ml or about from 34 to 36 μg/ml;
b) with a treatment regime of continuous infusion of Annexin A5 to the subject, or one or more separate administrations, for example, once, twice, three, four or more times daily;
c) is administered in a dosage amount at each administration in the range of from about 5 to 20 mg/kg patient body weight, such as from about 10 to 15 mg/kg, such as about 11 mg/kg, about 12 mg/kg, about 13 mg/kg or about 14 mg/kg;
d) at a total doses of Annexin A5 per administration in the region of for example, 0.1 to 3 g, such as 0.2 to 2 g, 0.5 to 1.5 g, 0.8 to 1.2 g or about lg of Annexin A5; and/or
e) continually, or separate repeated dosages, for a period of at least, or up to, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 day, 4 weeks, 5 weeks, 6 weeks, 7 weeks 8 weeks, 3 months, 4 months, 5 months or longer.

29. The method according to claim 1, wherein the Annexin A5 is administered to the subject by injection (such as intravenous injection) or infusion (such as intravenous infusion).

30. The method according to claim 1, wherein the subject has been in contact with biological material present in or taken from another subject infected or suspected of being infected with, a virus (such as a VHF), and wherein biological material has, or is suspected to have been in contact with an abrasion in the skin of the subject, the mucosal tissue of the subject and/by parenteral exposure to the subject.

31. The method according to claim 1, wherein the subject has been in contact with biological material present in or taken from another subject infected or suspected of being infected with, a virus (such as a VHF), and the contact occurred within the preceding 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

32. The method according to claim 1 wherein the subject to be treated is a subject suspected of being infected with a virus as defined by any one of claims 1 to 21.

33. The method according to claim 32 wherein the subject suspected of being infected displays one or more symptoms of infection with the virus.

34. The method according to claim 1, wherein the subject has been diagnosed with, and has recovered from, an infection with the virus.

35. The method according to claim 34, wherein the subject has been recovered from an infection with the virus for a period of time of about, up to, or at least, 1, 2, 3, 4, 5, 6 or 7 days, 1, 2, 3 or 4 weeks, 1, 2, 3, 4, 5, 6, 7, 8, 9, 10, 11, or 12 months, 1, 2, 3, 4, 5, 6, 7, 8, 9 or 10 years or more.

36. The method according to claim 1, wherein the symptoms of infection by VHF (such as Ebola) detectable in the subject include one or more symptoms selected from initial clinical symptoms, such as excessive or profuse sweating, the onset of fever, myalgia, general malaise, and/or chills; and/or flu-like symptoms optionally accompanied by gastro-intestinal symptoms; maculo-papulary rash, petichae, conjunctival hemorrhage, epistaxis, melena, hematemesis, shock and/or encephalopathy; leukopenia (for example, associated with increased lymphoid cell apoptosis), thrombocytopenia, increased levels of aminotransferase, thrombin and/or partial thromboplastin times, fibrin split products detectable in the blood, and/or disseminated intravascular coagulation (DIC).

37. The method according to claim 1, wherein the subject is a human, or a non-human animal, including an animal selected from the group consisting of dogs, cats, horses, cattle, sheep, pigs, goats, rodents, camels, birds, insects, domesticated animals, and wild animals.

38. The method according to claim 1, wherein the subject is a human, such as a human health worker, in particular a health worker who works or has worked with patients having, or being suspect of having, an infection with a virus, for example a VHF, such as Ebola virus, or a virus that presents phosphatidylserine (PS) and mediates cell infection and/or intcrnalisationinternalization through PS binding.

39. The method according to claim 1, wherein the subject is a family member of, and/or shares or shared accommodation with, a patient having, or being suspect of having, an infection with a virus, for example a VHF, such as Ebola virus, or a virus that presents phosphatidylserine (PS) and mediates cell infection and/or internalization through PS binding.


SEQUENCE LISTING (2 sequences)

SEQ ID NO: 1 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 2 - protein, 362 aa, Homo sapiens
MHPQVVILSLILHLADSVAGSVKVGGEAGPSVTLPCHYSGAVTSMCWRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNQTQLFLEHSLLTANTTKGIYAGVCISVLVLLALLGVIIAKKYFFKKEVQQLSVSFSSLQIKALQNAVEKEVQAEDNIYIENSLYATD